(19) United States
(12) Patent Application Publication
(10) Pub. No.: US 2024/0010723 A1
(43) Pub. Date: Jan. 11, 2024

(54) ANTIBODIES TARGETED TO CD147

(71) Applicant: Ibex Biosciences, Inc., Cumberland, MD (US)

(72) Inventors: Vidal Felix de la CRUZ, Phoenixville, PA (US); Alberto Murat Croci, Washington, DC (US); Michael Joseph Karlin, Bethesda, MD (US)

(21) Appl. No.: 18/347,295

(22) Filed: Jul. 5, 2023

Related U.S. Application Data
(63) Continuation of application No. PCT/US2022/011969, filed on Jan. 11, 2022.
(60) Provisional application No. 63/135,827, filed on Jan. 11, 2021.

(51) Int. Cl. C07K 16/28; A61P 35/00; A61P 33/06; A61K 47/68

(52) U.S. Cl. CPC C07K 16/2803; A61P 35/00; A61P 33/06; A61K 47/6849; A61K 2039/505

(57) ABSTRACT

Provided herein are novel antibody sequences that bind to CD147 and inhibit viral or parasite invasion, reduce inflammation, and reduce cancer cell viability. The present disclosure also provides methods of using these antibodies to treat viral infection and cancer.

### CROSS REFERENCE TO RELATED APPLICATIONS

This application is a continuation of International Application No. PCT/US2022/011969, filed Jan. 11, 2022, which claims priority to U.S. Provisional Patent Application No. 63/135,827 filed Jan. 11, 2021, the entire contents of each of which are incorporated by reference herein.

### DESCRIPTION OF THE TEXT FILE SUBMITTED ELECTRONICALLY

The contents of the electronic sequence listing (IBEX_005_01US_SeqList_ST26.xml; Size: 56,924 bytes; and Date of Creation: Jul. 5, 2023) are herein incorporated by reference in its entirety.

### INCORPORATION BY REFERENCE STATEMENT

All publications, patents, and patent publications cited are incorporated by reference herein in their entirety for all purposes.

### BACKGROUND

The extracellular matrix metalloproteinase inducer CD147 (also known as Basigin, EMMPRIN, Neurothelin) is a transmembrane glycoprotein that belongs to the immunoglobulin superfamily. CD147 is involved in tumor development, plasmodium invasion and virus infection. Furthermore, CD147 has been investigated as a potential therapeutic target for cancer and immunological disorders such as rheumatoid arthritis.

### BRIEF SUMMARY

The present disclosure provides novel antibodies, antibody fragments, or modifications thereof that bind to CD147 expressed on cells to prevent viral infection, inhibit malaria infection, inhibit angiogenesis, and decrease cell viability.

Provided herein is an anti-CD147 antibody comprising a binding domain that comprises at least one CDR comprising at least 70% identity to an amino acid sequence selected from SEQ ID NOs: 16-39. In some embodiments, the anti-CD147 antibody comprises two CDRs, wherein each of the two CDRs independently comprise at least 70% identity to an amino acid sequence selected from SEQ ID NOs: 16-39. In some embodiments, the anti-CD147 antibody comprises three CDRs, wherein each of the three CDRs independently comprise at least 70% identity to an amino acid sequence selected from SEQ ID NOs: 16-39. In some embodiments, the anti-CD147 antibody comprises an amino acid sequence with at least 70% identity to a sequence selected from SEQ ID NOs: 4-15. In some embodiments, the anti-CD147 antibody comprises an amino acid sequence selected from SEQ ID NOs: 4-15. In some embodiments, the antibody is a full-length antibody, a monospecific antibody, a bispecific antibody, a trispecific antibody, an antigen-binding region, heavy chain, light chain, VhH, Vh, a CDR, a variable domain, scFv, Fc, Fv, Fab, F(ab)2, IgG, reduced IgG (rIgG), monospecific Fab2, bispecific Fab2, trispecific Fab3, diabody, bispecific diabody, trispecific triabody, minibody, nanobody, IgNAR, V-NAR, HcIgG, or a combination thereof. In some embodiments, the antibody is the VhH. In some embodiments, a binding region of a Chimeric Antigen Receptor (CAR) comprises at least one CDR comprising at least 70% identity to an amino acid sequence selected from SEQ ID NOs: 16-39. In some embodiments, the CAR is expressed on an immune cell. In some embodiments, the immune cell is a PBMC, a lymphocyte, a T cell, or a NK cell. In some embodiments, the anti-CD147 antibody binds to a CD147 or fragment thereof expressed on the surface of a cell. In some embodiments, the cell is an epithelial cell, an endothelial cell, or neuronal cell. In some embodiments, the anti-CD147 antibody reduces or eliminates interaction between a virus and CD147. In some embodiments, the interaction comprises binding of the CD147 by the virus. In some embodiments, the anti-CD147 antibody reduces or eliminates a viral spike protein from binding to CD147. In some embodiments, the anti-CD147 antibody reduces or eliminates viral invasion of a cell. In some embodiments, the anti-CD147 antibody reduces or eliminates viral invasion of a cell by at least about 1-fold as determined by a viral infectivity assay. In some embodiments, the virus is selected from the group consisting of: measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g., HIV), enveloped virus, RNA virus, and hepatitis. In some embodiments, the virus is the coronavirus. In some embodiments, the coronavirus is SARS-CoV-2. In some embodiments, the cell is a cancer cell. In some embodiments, the cell is a tumor cell. In some embodiments, the cancer cell is from a hematological cancer. In some embodiments, the tumor cell is from a cancer selected from the group consisting of: breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL). In some embodiments, the anti-CD147 antibody reduces or eliminates tumor cell proliferation, metastasis, secretion of matrix metalloproteinases, degradation of a tumor matrix, tumor cell invasion, and/or angiogenesis. In some embodiments, the anti-CD147 antibody reduces or eliminates inflammation. In some embodiments, the anti-CD147 antibody is effective in reducing or eliminating an inflammatory or autoimmune disorder. In some embodiments, the inflammatory or autoimmune disease is selected from the group consisting of: rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis. In some embodiments, the anti-CD147 antibody is humanized. In some embodiments, the anti-CD147 antibody binds to a cell thereby preventing, inhibiting, or decreasing invasion of a Plasmodium parasite into the cell. In some embodiments, the invasion of the Plasmodium parasite into the cell is by at least about 1-fold. In some embodiments, the cell is a red blood cell. In some embodiments, the Plasmodium parasite is selected from the group consisting of: Plasmodium falciparum, Plasmodium malariae, Plasmodium ovale, Plasmodium vivax, and Plasmodium knowlesi.

In some embodiments, the anti-CD147 antibody is effective in decreasing parasitemia in blood cells. In some embodiments, the anti-CD147 antibody decreases parasitemia in blood cells within about 48 hours. In some embodiments, the anti-CD147 antibody decreases parasitemia in blood cells within about 96 hours. In some embodiments, administration of the anti-CD147 antibody prevents, ameliorates, or treats malaria in a subject.

Provided herein is also a method of preventing, treating, or ameliorating a disorder associated with CD147 expression in a subject in need thereof comprising administering an anti-CD147 antibody.

Provided herein is also a pharmaceutical composition comprising an effective amount of an anti-CD147 antibody.

Provided herein is also a cell comprising a sequence encoding an anti-CD147 antibody.

Provided herein is an anti-CD147 antibody that comprises a CDR1, a CDR2, and a CDR3 region, wherein the CDR1 region is an amino acid sequence selected from the group consisting of: SEQ ID NO: 12-SEQ ID NO: 15, wherein the CDR2 region is an amino acid sequence selected from the group consisting of: SEQ ID NO: 16-SEQ ID NO: 19, wherein the CDR3 region is an amino acid sequence selected from the group consisting of: SEQ ID NO: 20-SEQ ID NO: 23, and wherein the anti-CD147 antibody comprises from 0 to 5 amino acid modifications in at least one of the CDR1, CDR, or CDR3 regions. In some embodiments, the anti-CD147 antibody comprises from 0-3 amino acid modifications. In some embodiments, the CDR1 region corresponds to SEQ ID NO: 18 or 22, wherein the CDR2 region corresponds to SEQ ID NO: 26 or 30, and wherein the CDR3 region corresponds to SEQ ID NO: 34 or 38. In some embodiments, the anti-CD147 antibody comprises SEQ ID NO: 12 or SEQ ID NO: 14. In some embodiments, the anti-CD147 antibody is humanized.

Provided herein is a method of treatment, comprising administering an effective amount of a pharmaceutical composition comprising an anti-CD147 antibody, wherein the anti-CD147 antibody comprises a CDR1, a CDR2, and a CDR3 region, wherein the CDR1 region is an amino acid sequence selected from the group consisting of: SEQ ID NO: 16-SEQ ID NO: 23, wherein the CDR2 region is an amino acid sequence selected from the group consisting of: SEQ ID NO: 24-SEQ ID NO: 31, wherein the CDR3 region is an amino acid sequence selected from the group consisting of: SEQ ID NO: 32-SEQ ID NO: 39, and wherein the anti-CD147 antibody comprises from 0 to 2 amino acid modifications in at least one of the CDR1, CDR, or CDR3 regions. In some embodiments, the administering is effective in reducing or eliminating an inflammatory or autoimmune disease. In some embodiments, the inflammatory or autoimmune disease is selected from the group consisting of: rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis. In some embodiments, the administering is effective in reducing or eliminating viral invasion of a cell by a virus. In some embodiments, the virus is selected from the group consisting of: measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g., HIV), enveloped virus, RNA virus, and hepatitis. In some embodiments, the virus is the coronavirus. In some embodiments, the coronavirus comprises SARS-CoV-2. In some embodiments, the administering is effective in reducing or eliminating metastasis of a cancer. In some embodiments, the cancer is selected from the group consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL). In some embodiments, the administering is effective in reducing or eliminating invasion of Plasmodium into a cell.

Provided herein is an antibody-drug conjugate that comprises an anti-CD147 antibody. In some embodiments, the drug is selected from the group consisting of an anti-cancer drug, an anti-malaria drug, an anti-viral drug, a toxin, and an anti-inflammatory drug. In some embodiments, the drug comprises the anti-viral drug, wherein the anti-viral drug is an anti-SARS-CoV-2 drug. In some embodiments, the drug comprises the anti-cancer drug, wherein the anti-cancer drug comprises an immunotherapy. In some embodiments, the immunotherapy is selected from the group consisting of an antibody, checkpoint inhibitor, cell therapy, cytokine, oncolytic virus, and vaccine. In some embodiments, the ADC comprises an antibody. In some embodiments, the antibody comprises SEQ ID NO: 12 or SEQ ID NO: 14.

In some embodiments, the present disclosure provides an anti-CD147 antibody, antibody fragment, or modification thereof that binds to a CD147 polypeptide. In some embodiments, the present disclosure provides an anti-CD147 antibody or fragment thereof comprising an amino acid sequence having at least one CDR comprised in any of SEQ ID NOs: 16-39. In some embodiments, the antibody comprises two to six CDRs comprised in any of SEQ ID NOs: 16-39. In some embodiments, the antibody comprises all the CDRs comprised in any one of SEQ ID NOs: 16-39. In some embodiments, the antibody comprises an amino acid sequence at least 70% identical to any of SEQ ID NOs: 16-39. In some embodiments, the antibody comprises an amino acid sequence of any of SEQ ID NOs: 16-39.

In some embodiments, the anti-CD147 antibody is a full-length antibody, a monospecific antibody, a bispecific antibody, a trispecific antibody, an antigen-binding region, heavy chain, light chain, VhH, Vh, Vl, a CDR, a variable domain, scFv, Fc, Fv, Fab, F(ab)2, reduced IgG (rIgG), monospecific Fab2, bispecific Fab2, trispecific Fab3, diabody, bispecific diabody, trispecific triabody, minibody, nanobody, IgNAR, V-NAR, HcIgG, or a combination thereof. In some embodiments, the anti-CD147 antibody is a VhH. In some embodiments, the anti-CD147 antibody is comprised in a Chimeric Antigen Receptor (CAR). In some embodiments, the CAR is expressed from an immune cell. In some embodiments, the immune cell is a PBMC, a lymphocyte, a T cell, or a NK cell.

In some embodiments, the anti-CD147 antibody, antibody fragment, or modification binds to a CD147 polypeptide or fragment thereof expressed on the surface of a cell. In some embodiments, the cell is an epithelial, endothelial, or neuronal cell. In some embodiments, the cell is a tumor cell. In some embodiments, the tumor cell is from a solid tumor. In some embodiments, the tumor cell is from a hematological cancer. In some embodiments, the tumor cell is from a cancer consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

In some embodiments, the anti-CD147 antibody, antibody fragment, or modification inhibits interaction of a virus and CD147. In some embodiments, the anti-CD147 antibody prevents or reduces a virus from binding to CD147. In some embodiments, the anti-CD147 antibody prevents or reduces a viral spike protein from binding to CD147.

In some embodiments, the anti-CD147 antibody reduces or eliminates viral invasion of a cell. In some embodiments, the anti-CD147 antibody prevents or reduces viral infection. In some embodiments, the virus is selected from the group consisting of measles, coronavirus, SARS, MERS, SARS-CoV-2, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis. In some embodiments, the virus is a coronavirus. In some embodiments, the coronavirus is SARS-CoV-2.

In some embodiments, the anti-CD147 antibody, antibody fragment, or modification thereof prevents or reduces tumor cell proliferation, metastasis, the secretion of matrix metalloproteinases, the degradation of the matrix, tumor cell invasion, and/or angiogenesis.

In some embodiments, the anti-CD147 antibody, antibody fragment, or modification thereof prevents or reduces inflammation. In some embodiments, the antibody treats or ameliorates an inflammatory or autoimmune disorder. In some embodiments, the inflammatory or autoimmune disease is selected from rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

In some embodiments, the present disclosure provides a method of preventing, treating, or ameliorating a disorder associated with CD147 expression in a subject in need thereof comprising administering the anti-CD147 antibody, antibody fragment, or modification thereof described herein.

In some embodiments, the present disclosure provides a pharmaceutical composition comprising the anti-CD147 antibody, antibody fragment, or modification thereof described herein.

In some embodiments, the present disclosure provides a cell comprising a sequence encoding the anti-CD147 antibody, antibody fragment, or modification thereof described herein.

### BRIEF DESCRIPTION OF THE DRAWINGS

FIG. 1 shows an ELISA demonstrating the binding of anti-CD147 antibodies of the disclosure to CD147-coated plates.

FIG. 2 shows that none of the tested antibodies bind to murine (mouse) mCD147.

FIG. 3 shows a comparison of the binding of the tested antibodies to human and murine CD147.

FIG. 4 shows a steady state model of Ibx-77 binding to CD147.

FIG. 5 shows a kinetic model of Ibx-77 binding to CD147.

FIG. 6 shows a Kd/Ka kinetic model of Ibx-77 binding to CD147.

FIG. 7 shows a steady state 1:1 fitting model of Ibx-77 binding to CD147.

FIG. 8 shows Ibx-77 staining of HEK-293 cells.

FIG. 9 shows Ibx-77 staining of HCC-1954 cells.

FIG. 10 shows Ibx-77 staining of HeLa cells.

FIGS. 11A-B show Ibx-11, Ibx-13, and Ibx-77 staining of HCC-1954 cells.

FIGS. 12A-B show Ibx-11, Ibx-13, and Ibx-77 staining of HeLa cells.

FIG. 13 shows Ibx-11 staining of Jurkat cells.

FIGS. 14A-B show Ibx-11 and Ibx-13 staining of Vero E6 cells.

FIGS. 15A-B show sensograms of Ibx-75 (A) and Ibx-74 (B).

FIG. 16 shows that Ibx-11 and Ibx-13 antibody-drug conjugates (ADCs) effectively kill MiaPaCa-2 cancer cells.

FIG. 17 shows difference in the pattern of aggregation between Ibx-series treated cells and control cells.

FIG. 18 shows imaging analysis measuring pixel size: 50 μg/ml-25 μg/ml Ibx-series antibodies show larger pixel sizes for the particles (aggregates) at 24 hours.

FIG. 19 shows images from this Jurkat aggregation assay counted at 18 hours and 24 hours.

FIG. 20 shows that at 24 hours, there was a significant decrease in single cell counts for Ibx-77 treated cells.

FIG. 21 demonstrates cells treated with either Ibx-76 or Ibx-77 showed an inhibition of angiogenesis in a tube-forming assay.

FIG. 22 shows the percent parasitemia in vitro after administration of Ibx-13 (AB1), MIF-2-5 (AB2), or VhH-Fc (AB3) at 48 and 96 hours.

FIG. 23A-B show the percent parasitemia in vitro after administration of Ibx-13, MF22.3 (AB2) and h378GL (AB3) at 1 μg/mL, 10 μg/mL, and 100 μg/mL. FIG. 23A shows percent parasitemia at 48 hours after administration; FIG. 23B shows percent parasitemia after 96 hours.

FIG. 24 shows A375 malignant melanoma cell viability after treatment with varying concentrations of Ibx-11, Ibx-13, or control antibodies.

FIG. 25 shows a comparison of A375 malignant melanoma cell viability after treatment with 30 nM of Ibx-11, Ibx-13, or control antibodies.

FIG. 26 shows PC3 prostate cancer cell viability after treatment with varying concentrations of Ibx-11, Ibx-13, or control antibodies.

FIG. 27 shows a comparison of PC3 prostate cancer cell viability after treatment with 30 nM of Ibx-11, Ibx-13, or control antibodies.

### DETAILED DESCRIPTION

### CD147 in Cancer

CD147 is a highly glycosylated transmembrane protein of the immunoglobulin super family that acts as the main upstream stimulator of matrix metalloproteinases (MMPs). Expression levels of CD147 and MMPs are often increased in inflammatory processes and tumors and are associated with cancer progression. Cancer stem cells characterized by high cell surface CD147 expression produce more hyaluronan, resulting in stabilization of lipid rafts, as well as enhanced MMP and ABC drug transporter expression, resistance, and survival.

In some embodiments, anti-CD147 antibodies or fragments, or modifications thereof described herein can be used to treat or prevent the growth of a cancer. In some embodiments, the anti-CD147 antibodies or fragments, or modifications thereof, and modified versions (e.g., Antibody-Drug Conjugates ADCs) treat or prevent the growth of a hematologic cancer. In some embodiments, the anti-CD147 antibodies or fragments, or modifications thereof treat or prevent the growth of a solid cancer. In some embodiments, the anti-CD147 antibodies or fragments, or modifications thereof treat or prevent the growth of a hematological cancer. In some embodiments, the anti-CD147 antibodies or fragments, or modifications thereof treat or prevent the growth of a metastatic cancer. In some embodiments, the cancer includes, but is not limited to, breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

### Autoimmune and Inflammatory Disorders

The anti-CD147 antibodies, fragments, or modifications thereof may be used to treat or reduce inflammation in a subject. In some embodiments, the inflammation is associated with an autoimmune or inflammatory disorder. In some embodiments, the autoimmune or inflammatory disorder includes, but is not limited to, rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

### Viral Infections

The anti-CD147 antibodies, fragments, or modifications thereof may be used to treat or reduce viral infection and/or viral load in a subject. In some embodiments, the anti-CD147 antibodies, fragments, or modifications thereof may be used to prevent, inhibit, or reduce viral invasion into a cell. CD147 is known to be important for viral invasion of cells. For example, CD147 is known to be a functional entry receptor for measles virus invasion of epithelial cells Watanabe 2010. Further, during HIV-1 infection, the CD147 molecule can promote infection of the host cell by the HIV-1 virus through interaction with the virus-associated CyPA (Chenglong 2020). In some embodiments, the viral infection is caused by a virus including, but not limited to, measles, coronavirus, SARS, MERS, SARS-CoV-2, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis.

### Malaria Infections

The anti-CD147 antibodies, fragments, or modifications thereof may be used to treat or reduce the symptoms of malaria in a subject. In some embodiments, the anti-CD147 antibodies, fragments, or modifications thereof may be used to prevent, inhibit, or reduce invasion of one or more Plasmodium strains into a cell. In some embodiments, the cell is a red blood cell. In some embodiments, the Plasmodium strain is selected from Plasmodium falciparum, Plasmodium malariae, Plasmodium ovale, Plasmodium vivax, and Plasmodium knowlesi. In some embodiments, the anti-CD147 antibodies, fragments, or modifications thereof prevent the invasion of Plasmodium falciparum: into a cell. In some embodiments, the anti-CD147 antibodies, fragments, or modifications thereof prevent the invasion of multiple Plasmodium strains into a cell. In some embodiments, the Plasmodium is Plasmodium falciparum. In some embodiments, the Plasmodium is Plasmodium malariae.

In some embodiments, the anti-CD147 antibody or fragment or modification thereof decreases parasitemia. In some embodiments, the anti-CD147 antibody or fragment or modification thereof decreases parasitemia. in blood cells. In some embodiments, the anti-CD147 antibody or fragment or modification thereof decreases parasitemia. in blood cells within 10 minutes to 120 hours. In some embodiments, the anti-CD147 antibody or fragment or modification thereof decreases parasitemia. in blood cells within about 10 minutes, about 20 minutes, about 30 minutes, about 40 minutes, about 50 minutes, about 1 hour, about 2 hours, about 3 hours, about 4 hours, about 5 hours, about 6 hours, about 7 hours, about 8 hours, about 9 hours, about 10 hours, about 12 hours, about 15 hours, about 20 hours, about 24 hours, about 25 hours, about 30 hours, about 35 hours, about 36 hours, about 40 hours, about 45 hours, about 48 hours, about 50 hours, about 55 hours, about 60 hours, about 65 hours, about 70 hours, about 75 hours, about 80 hours, about 85 hours, about 90 hours, about 95 hours, about 96 hours, about 100 hours, about 105 hours, about 110 hours, about 115 hours, or about 120 hours. In some embodiments, the anti-CD147 antibody or fragment or modification thereof decreases parasitemia. in blood cells within about 48 hours. In some embodiments, the anti-CD147 antibody or fragment or modification thereof decreases parasitemia. in blood cells within about 96 hours. In some embodiments, the blood cells are red blood cells.

### SARS-CoV-2

As of January 2022, SARS-CoV-2 has infected more than 293 million people (>55 million in the USA), has claimed over 5.4 million lives worldwide (>821, 408 in the USA) and continues to spread at an alarming rate without any signs of slowing down. One of the factors that contributes to the high virulence and pathogenicity of SARS-CoV-2 is its ability to infect multiple tissues resulting in organ failure and death in severe cases. Similar to other enveloped viruses, SARS-CoV-2 utilizes host-cell receptors to invade host cells ultimately resulting in tissue necrosis, organ damage, and eventually death. CD147 is one such host cell receptor that facilitates viral entry.

Angiotensin-converting enzyme 2 (ACE2) is generally recognized as the primary receptor for SARS-CoV-2 infection but other molecules are being reported as significantly contributing to the cell tropism for this virus. CD147 has recently garnered attention for its role in SARS-CoV-2 cell invasion via direct interaction with the Spike protein Recognition Binding Domain (RBD) of the virus, with potential benefits observed in an open label clinical trial that demonstrated early resolution of COVID-19 symptoms in patients who were treated with anti-CD147 antibody.

### Anti-CD147 Antibodies

In some embodiments, the present disclosure provides novel antibodies that bind to CD147 and their use in preventing or ameliorating a condition or disease. In some embodiments, the CD147 is expressed on the surface of a cell. In some embodiments, anti-CD147 antibodies of the disclosure can exert their effect directly or indirectly. In some embodiments, an anti-CD147 antibody binds to CD147 thereby directly reducing or eliminating binding of the CD147 by anything else (e.g., a virus or cancer cell). In some embodiments, an anti-CD147 antibody can sterically hinder binding of a CD147 receptor thereby indirectly exerting its effect. In some embodiments, an anti-CD147 can deliver a blocking agent when complexed as part of an antibody-drug conjugate thereby indirectly reducing or eliminating binding to a CD147 receptor. Any of the anti-CD147 antibodies can be fully human, humanized, murine, rat, rabbit, and any combination thereof. In some embodiments, an anti-CD147 antibody is humanized. In some embodiments, an anti-CD147 antibody is fully human.

In some embodiments, the anti-CD147 antibody binds to a human CD147 polypeptide, isoform, or fragment thereof. In some embodiments, the human CD147 is an isoform. In some embodiments, the CD147 isoform is basigin or basigin-2, which has two immunoglobulin domains. In some embodiments, the CD147 isoform is basigin-1, which has three immunoglobulin domains. In some embodiments, the human CD147 polypeptide or fragment thereof is a polypeptide comprising the amino acid sequence of SEQ ID NO: 1. In some embodiments, the human CD147 polypeptide or fragment thereof is a polypeptide comprising at least about 70% sequence identity to SEQ ID NO: 1. In some embodiments, the human CD147 polypeptide or fragment thereof is a polypeptide comprising at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about or about 99.9% to SEQ ID NO: 1. In some embodiments, the CD147 polypeptide, isoform, or fragment thereof is expressed on the surface of a cell. In some embodiments, the sequence of the portion of the CD147 polypeptide, isoform, or fragment thereof involved in binding is not altered. In some embodiments, the sequence of the portion of the CD147 polypeptide, isoform, or fragment thereof involved in binding is altered.

In some embodiments, the human CD147 polypeptide or fragment is a truncation of the polypeptide comprising the amino acid sequence of SEQ ID NO: 1. In some embodiments, the polypeptide is truncated at the N-terminus. In some embodiments, the polypeptide is truncated at the C-terminus. In some embodiments, the polypeptide is truncated at both the N- and C-termini. In some embodiments, the polypeptide is truncated by up to about 20 amino acids compared to the polypeptide of SEQ ID NO: 1. In some embodiments, the polypeptide is truncated by about 20, about 19, about 18, about 17, about 16, about 15, about 14, about 13, about 12, about 11, about 10, about 9, about 8, about 7, about 6, about 5, about 4, about 3, about 2, or about 1 amino acid compared to the polypeptide of SEQ ID NO: 1.

In some embodiments, the anti-CD147 antibody binds to a rodent CD147 polypeptide or fragment thereof. In some embodiments, the rodent CD147 polypeptide or fragment thereof is a rat CD147. In some embodiments, the rodent CD147 polypeptide or fragment thereof is a murine CD147. In some embodiments, the murine CD147 polypeptide is a polypeptide comprising the amino acid sequence of SEQ ID NO: 2. In some embodiments, the human CD147 polypeptide is a polypeptide comprising at least about 70% sequence identity to SEQ ID NO: 2. In some embodiments, the human CD147 polypeptide is a polypeptide comprising at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about or about 99.9% to SEQ ID NO: 2. In some embodiments, the sequence of the portion of the polypeptide involved in binding CD147 is not altered. In some embodiments, the sequence of the portion of the polypeptide involved in binding CD147 is altered.

In some embodiments, the murine CD147 polypeptide or fragment is a truncation of the polypeptide comprising the amino acid sequence of SEQ ID NO: 2. In some embodiments, the polypeptide is truncated at the N-terminus. In some embodiments, the polypeptide is truncated at the C-terminus. In some embodiments, the polypeptide is truncated at both the N- and C-termini. In some embodiments, the polypeptide is truncated by up to about 20 amino acids compared to the polypeptide of SEQ ID NO: 2. In some embodiments, the polypeptide is truncated by about 20, about 19, about 18, about 17, about 16, about 15, about 14, about 13, about 12, about 11, about 10, about 9, about 8, about 7, about 6, about 5, about 4, about 3, about 2, or about 1 amino acid compared to the polypeptide of SEQ ID NO: 2.

In some embodiments, the CD147 is expressed on an endothelial cell, epithelial cell, neuronal cell, immunological cell, blood cell, fibroblast, T-cell, Peripheral Blood Mononuclear Cell (PBMC), monocyte cell, or mononuclear cell. In some embodiments, the CD147 is aberrantly expressed on the surface of a cell. In some embodiments, the CD147 is over-expressed on the surface of a cell. In some embodiments, the CD147 is expressed by a cell of a tumor stroma.

In some embodiments, the antibody that binds to a CD147 polypeptide or fragment thereof is a full length antibody, a monospecific antibody, a bispecific antibody, a trispecific antibody, an antigen-binding region, heavy chain, light chain, VhH, Vh, Vl, a CDR, a variable domain, scFv, Fc, Fv, Fab, F(ab)2, reduced IgG (rIgG), monospecific Fab2, bispecific Fab2, trispecific Fab3, diabody, bispecific diabody, trispecific triabody, minibody, nanobody, IgNAR, V-NAR, HcIgG, or a combination thereof.

In some embodiments, any of the described anti-CD147 antibodies may comprise one or more linkers, tags, or both linkers and tags. For example, any of the anti-CD147 antibody fusion constructs may be connected by way of one or more linkers, see for example fusion constructs in Table 1. In some embodiments, a linker is a cleavage linker. In some embodiments, a linker is not cleavable. A linker of the disclosure may be protease sensitive, pH sensitive, and/or glutathione sensitive. A cleavable linker may be utilized to release a drug more effectively at a target site (e.g., at a tumor). Exemplary cleavable linkers include but are not limited to: hydrazone, SPDB, SPP, vc, va, and combinations thereof. Exemplary non-cleavable linkers comprise: mcc and mc. In some embodiments, flexible linkers may be utilized in fusion constructs, for example a (GGGGS)₃ (SEQ ID NO: 40) linker. Another linker that may be utilized comprises the EPKIPQPQPKPQPQPQPQPQPKPQPEP (SEQ ID NO: 41) sequence. In some embodiments, a tag may be utilized. An exemplary tag may comprise a flag tag such as DYKDDDDK (SEQ ID NO: 42).

In some embodiments, the antibody that binds to a CD147 polypeptide or fragment thereof comprises a VhH antibody. In some embodiments, the antibody that binds to a CD147 polypeptide or fragment thereof is a VhH antibody. In some embodiments, the VhH antibody is selected from any of SEQ ID NOs: 4-15. In some embodiments, the VhH antibody comprises at least about 70% sequence identity to the sequence of any of SEQ ID NOs: 4-15. In some embodiments, the VhH antibody comprises at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about or about 99.9% to the sequence of any of SEQ ID NOs: 4-15.

In some embodiments, the anti-CD147 antibody comprises one or more CDRs found in one or more of any of SEQ ID NOs: 4-15. In some embodiments, the anti-CD147 antibody comprises one CDR found in one or more of any of SEQ ID NOs: 4-15. In some embodiments, the anti-CD147 antibody comprises two CDRs found in one or more of any of SEQ ID NOs: 4-15. In some embodiments, the anti-CD147 antibody comprises two CDRs found in different sequences of any of SEQ ID NOs: 4-15. In some embodiments, the anti-CD147 antibody comprises three CDRs found in one or more of any of SEQ ID NOs: 4-15. In some embodiments, the anti-CD147 antibody comprises three CDRs found in one or more different sequences of any of SEQ ID NOs: 4-15.

TABLE 1
Exemplary Antibody Constructs and Fusions Comprising the Same

| | SEQ ID | |
|---|---|---|
| | NO: | Sequence |
| Human CD147 | 1 | MAAALFVLLGFALLGTHGASGAAGFVQAPLSQQRWVGG |
| (Uniprot | | SVELHCEAVGSPVPEIQWWFEGQGPNDTCSQLWDGARL |
| P35613) | | DRVHIHATYHQHAASTISIDTLVEEDTGTYECRASNDPDR |
| | | NHLTRAPRVKWVRAQAVVLVLEPGTVFTTVEDLGSKILL |
| | | TCSLNDSATEVTGHRWLKGGVVLKEDALPGQKTEFKVD |
| | | SDDQWGEYSCVFLPEPMGTANIQLHGPPRVKAVKSSEHI |
| | | NEGETAMLVCKSESVPPVTDWAWYKITDSEDKALMNGS |
| | | ESRFFVSSSQGRSELHIENLNMEADPGQYRCNGTSSKGSD |
| | | QAIITLRVRSHLAALWPFLGIVAEVLVLVTIIFIYEKRRKPE |
| | | DVLDDDDAGSAPLKSSGQHQNDKGKNVRQRNSS |
| Murine CD147 | 2 | MAAALLLALAFTLLSGQGACAAAGFLKAPLSQERWAGG |
| (Uniprot | | SVVLHCEAVGSPIPEIQWWFEGNAPNDSCSQLWDGARLD |
| P18572) | | RVHIHAAYRQHAASSLSVDGLTAEDTGTYECRASSDPDR |
| | | NHLTRPPRVKWVRAQASVVVLEPGTIQTSVQEVNSKTQL |
| | | TCSLNSSGVDIVGHRWMRGGKVLQEDTLPDLHTKYIVDA |
| | | DDRSGEYSCIFLPEPVGRSEINVEGPPRIKVGKKSEHSSEG |
| | | ELAKLVCKSDASYPPITDWFWFKTSDTGEEEAITNSTEAN |
| | | GKYVVVSTPEKSQLTISNLDVNVDPGTYVCNATNAQGTT |
| | | RETISLRVRSRMAALWPFLGIVAEVLVLVTIIFIYEKRRKP |
| | | DQTLDEDDPGAAPLKGSGTHMNDKDKNVRQRNAT |
| Rat CD147 | 3 | MAAALLLALAFTFLSGQGACAAAGFLKAPMSQEQWAGG |
| (Uniprot | | SVVLHCEAVGSPMPEIQWWFEGNEPNDSCSQLWDGARL |
| P26453) | | DRVHIHATYRQHAASTLSVDGLAAEDTGTYECRASSDPD |
| | | RNHLTRPPRVKWVRAQASVVVLEPGTIVTSVQEVDSKTQ |
| | | LTCFLNSSGIDIVGHRWMRGGKVLQEDTLPDLQMKYTV |
| | | DADDRSGEYSCIFLPEPVGRGNINVEGPPRIKVGKKSEHA |
| | | SEGEFVKLICKSEASHPPVDEWVWFKTSDTGDQTISNGTE |
| | | ANSKYVIISTPELSELIISDLDMNVDPGTYVCNATNSQGSA |
| | | RETISLRVRSRLAALWPFLGIVAEVLVLVTIIFIYEKRRKPD |
| | | QTLDEDDPGAAPLKGSGSHLNDKDKNVRQRNAT |

VhH Antibody-Fragment Crystallizable (Fc) Fusion Constructs

| | | |
|---|---|---|
| Ibx-11-Fc | 4 | QVRLEESGGGLVQAGGSLRLSCAASGLAFSIYGMGWFRQ |
| | | APGKEREFVSVVDNTGSTTLYADSVKGRFTISRDNAKNT |
| | | VYLQMSSLEPEDTAVYYCGAGPKNYLGSHEYAYWGQGT |
| | | QVTVSSGGGGSGGGGSGGGGSDKTHTCPPCPAPELLGGP |
| | | SVFLFPPKPKDTLMISRTPEVTCVVVDVSHEDPEVKFNWY |
| | | VDGVEVHNAKTKPREEQYNSTYRVVSVLTVLHQDWLNG |
| | | KEYKCKVSNKALPAPIEKTISKAKGQPREPQVYTLPPSRE |
| | | EMTKNQVSLTCLVKGFYPSDIAVEWESNGQPENNYKTTP |
| | | PVLDSDGSFFLYSKLTVDKSRWQQGNVFSCSVMHEALH |
| | | NHYTQKSLSLSPGK |
| Ibx-12-Fc | 5 | QVRLVESGGGLVQAGDSLRLSCQASGGTFTTYAMGWFR |
| | | QAPGQEREFVSATTWSTGTSYYAYAADSVKGRFTTSRDN |
| | | DNTVTLTMKRLKPDDTAVYYCSADPDGSDWAHSARQLR |
| | | YWGQGTQVTVSSGGGGSGGGGSGGGGSDKTHTCPPCPA |
| | | PELLGGPSVFLFPPKPKDTLMISRTPEVTCVVVDVSHEDPE |
| | | VKFNWYVDGVEVHNAKTKPREEQYNSTYRVVSVLTVLH |
| | | QDWLNGKEYKCKVSNKALPAPIEKTISKAKGQPREPQVY |
| | | TLPPSREEMTKNQVSLTCLVKGFYPSDIAVEWESNGQPEN |
| | | NYKTTPPVLDSDGSFFLYSKLTVDKSRWQQGNVFSCSVM |
| | | HEALHNHYTQKSLSLSPGK |
| Ibx-13-Fc | 6 | QVQLVESGGGLVQPGGSLRLSCTASEDTFSTYAIGWFRQ |
| | | APGQGREFVAGITANGIATRYNDSLKDRFTISRDNAKNTV |
| | | YLQMNSLQPEDTAVYSCGADTRRFSLPSTVHEIDYWGQG |
| | | TQVTVSSGGGGSGGGGSGGGGSDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSR |
| | | EEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| Ibx-14-Fc | 7 | QVQLVESGGGLVQAGDSLTLSCAASGRSLSSNAMGWFR |
| | | QAPGKEREFVSGTTWHAGTQYYAYAADSVKGRFTTSRD |
| | | NANTVSLQMKRLKPEDTAVYYCAAAVSGPGYDSWGQG |
| | | TQVTVSSGGGGSGGGGSGGGGSDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSR |
| | | EEMTKNQVSLTCLVKGFYPSDIAVEWESNGQPENNYKTT |
| | | PPVLDSDGSFFLYSKLTVDKSRWQQGNVFSCSVMHEALH |
| | | NHYTQKSLSLSPGK |
| Ibx-74 (dimer | 8 | QVRLEESGGGLVQAGGSLRLSCAASGLAFSIYGMGWFRQ |
| of Ibx-11) | | APGKEREFVSVVDNTGSTTLYADSVKGRFTISRDNAKNT |
| | | VYLQMSSLEPEDTAVYYCGAGPKNYLGSHEYAYWGQGT |
| | | QVTVSSEPKIPQPQPKPQPQPQPQPQPKPQPEPQVRLEESG |
| | | GGLVQAGGSLRLSCAASGLAFSIYGMGWFRQAPGKEREF |
| | | VSVVDNTGSTTLYADSVKGRFTISRDNAKNTVYLQMSSL |
| | | EPEDTAVYYCGAGPKNYLGSHEYAYWGQGTQVTVSS |
| Ibx-75 (dimer | 9 | QVRLVESGGGLVQAGDSLRLSCQASGGTFTTYAMGWFR |
| of Ibx-12) | | QAPGQEREFVSATTWSTGTSYYAYAADSVKGRFTTSRDN |
| | | DNTVTLTMKRLKPDDTAVYYCSADPDGSDWAHSARQLR |
| | | YWGQGTQVTVSSEPKIPQPQPKPQPQPQPQPQPKPQPEPQ |
| | | VRLVESGGGLVQAGDSLRLSCQASGGTFTTYAMGWFRQ |
| | | APGQEREFVSATTWSTGTSYYAYAADSVKGRFTTSRDND |
| | | NTVTLTMKRLKPDDTAVYYCSADPDGSDWAHSARQLRY |
| | | WGQGTQVTVSS |
| Ibx-76 (dimer | 10 | QVQLVESGGGLVQPGGSLRLSCTASEDTFSTYAIGWFRQ |
| of Ibx-13) | | APGQGREFVAGITANGIATRYNDSLKDRFTISRDNAKNTV |
| | | YLQMNSLQPEDTAVYSCGADTRRFSLPSTVHEIDYWGQG |
| | | TQVTVSSEPKIPQPQPKPQPQPQPQPQPKPQPEPQVQLVES |
| | | GGGLVQPGGSLRLSCTASEDTFSTYAIGWFRQAPGQGRE |
| | | FVAGITANGIATRYNDSLKDRFTISRDNAKNTVYLQMNS |
| | | LQPEDTAVYSCGADTRRFSLPSTVHEIDYWGQGTQVTVS |
| | | S |
| Ibx-77 (dimer | 11 | QVQLVESGGGLVQAGDSLTLSCAASGRSLSSNAMGWFR |
| of Ibx-14) | | QAPGKEREFVSGTTWHAGTQYYAYAADSVKGRFTTSRD |
| | | NANTVSLQMKRLKPEDTAVYYCAAAVSGPGYDSWGQG |
| | | TQVTVSSEPKIPQPQPKPQPQPQPQPQPKPQPEPQVQLVES |
| | | GGGLVQAGDSLTLSCAASGRSLSSNAMGWFRQAPGKER |
| | | EFVSGTTWHAGTQYYAYAADSVKGRFTTSRDNANTVSL |
| | | QMKRLKPEDTAVYYCAAAVSGPGYDSWGQGTQVTVSS |

Anti-CD147 VhH Antibody Constructs

| | | |
|---|---|---|
| Ibx-11 | 12 | QVRLEESGGGLVQAGGSLRLSCAASGLAFSIYGMGWFRQ |
| | | APGKEREFVSVVDNTGSTTLYADSVKGRFTISRDNAKNT |
| | | VYLQMSSLEPEDTAVYYCGAGPKNYLGSHEYAYWGQGT |
| | | QVTVSS |
| Ibx-12 | 13 | QVRLVESGGGLVQAGDSLRLSCQASGGTFTTYAMGWFR |
| | | QAPGQEREFVSATTWSTGTSYYAYAADSVKGRFTTSRDN |
| | | DNTVTLTMKRLKPDDTAVYYCSADPDGSDWAHSARQLR |
| | | YWGQGTQVTVSS |
| Ibx-13 | 14 | QVQLVESGGGLVQPGGSLRLSCTASEDTFSTYAIGWFRQ |
| | | APGQGREFVAGITANGIATRYNDSLKDRFTISRDNAKNTV |
| | | YLQMNSLOPEDTAVYSCGADTRRFSLPSTVHEIDYWGQG |
| | | TQVTVSS |
| Ibx-14 | 15 | QVQLVESGGGLVQAGDSLTLSCAASGRSLSSNAMGWFR |
| | | QAPGKEREFVSGTTWHAGTQYYAYAADSVKGRFTTSRD |
| | | NANTVSLQMKRLKPEDTAVYYCAAAVSGPGYDSWGQG |
| | | TQVTVSS |

In some embodiments, the anti-CD147 antibody comprises a CDR at least 70% identical to that of one or more CDRs found in any of SEQ ID NOs: 16-39. In some embodiments, the anti-CD147 antibody comprises one CDR at least about 70% identical to that of one or more CDRs found in one or more of any of SEQ ID NOs: 16-39. In some embodiments, the anti-CD147 antibody comprises two CDRs at least about 70% identical to one or more CDRs found in one or more of any of SEQ ID NOs: 16-39. In some embodiments, the anti-CD147 antibody comprises two CDRs at least about 70% identical to one or more CDRs found in different sequence of any of SEQ ID NOs: 16-39. In some embodiments, the anti-CD147 antibody comprises three CDRs at least about 70% identical to one or more CDRs found in one or more of any of SEQ ID NOs: 16-39. In some embodiments, the anti-CD147 antibody comprises three CDRs at least about 70% identical to one or more CDRs found in one or more different sequences of any of SEQ ID NOs: 16-39. In some embodiments, the one or more CDRs are at least about 60%, about 65%, about 70%, about 75%, 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about or about 99.9% to one or more CDRs found in one or more of any of SEQ ID NOs: 16-39.

In some embodiments, the anti-CD147 antibody comprises a CDR1 that comprises at least about 60% identity to a sequence selected from the group consisting of: SEQ ID NO: 16-23. In some embodiments, the anti-CD147 antibody comprises a CDR2 that comprises at least about 60% identity to a sequence selected from the group consisting of: SEQ ID NO: 24-31. In some embodiments, the anti-CD147 antibody comprises a CDR3 that comprises at least about 60% identity to a sequence selected from the group consisting of: SEQ ID NO: 32-39. In some embodiments, the anti-CD147 antibody comprises CDR1, CDR2, and CDR3 comprising SEQ ID NO: 16, 24, and 32 respectively. In some embodiments, the anti-CD147 antibody comprises CDR1, CDR2, and CDR3 comprising SEQ ID NO: 17, 25, and 33 respectively. In some embodiments, the anti-CD147 antibody comprises CDR1, CDR2, and CDR3 comprising SEQ ID NO: 18, 26, and 34 respectively. In some embodiments, the anti-CD147 antibody comprises CDR1, CDR2, and CDR3 comprising SEQ ID NO: 19, 27, and 35 respectively. In some embodiments, the anti-CD147 antibody comprises CDR1, CDR2, and CDR3 comprising SEQ ID NO: 20, 28, and 36 respectively. In some embodiments, the anti-CD147 antibody comprises CDR1, CDR2, and CDR3 comprising SEQ ID NO: 21, 29, and 37 respectively. In some embodiments, the anti-CD147 antibody comprises CDR1, CDR2, and CDR3 comprising SEQ ID NO: 22, 30, and 38 respectively. In some embodiments, the anti-CD147 antibody comprises CDR1, CDR2, and CDR3 comprising SEQ ID NO: 23, 31, and 39 respectively. In some embodiments, the anti-CD147 antibody comprises three CDR1, CDR2, and/or CDR3 sequences in tandem. In embodiments, an anti-CD147 antibody comprises, a CDR1, CDR2, or CDR3 in duplicate or triplicate, such as in any of the fusion constructs described herein. In some embodiments, a fusion construct may comprise any one of the CDR sequences described herein in tandem with one or more linkers.

TABLE 2
CDR Sequences of Exemplary anti-CD147 Constructs

| | SEQ ID | | SEQ ID | | SEQ ID | |
|---|---|---|---|---|---|---|
| | NO: | CDR1 | NO | CDR2 | NO | CDR3 |
| Ibx-11 | 16 | GLAFSIYG | 24 | VVDNTGS | 32 | GAGPKN |
| | | MG | | TTLYADS | | YLGSHEY |
| | | | | VKG | | AY |
| Ibx-12 | 17 | GGTFTTY | 25 | ATTWSTG | 33 | SADPDGS |
| | | AMG | | TSYYAYA | | DWAHSA |
| | | | | ADSVKG | | RQLRY |
| Ibx-13 | 18 | EDTFSTYA | 26 | GITANGIA | 34 | GADTRR |
| | | IG | | TRYNDSL | | FSLPSTV |
| | | | | KD | | HEIDY |
| Ibx-14 | 19 | GRSLSSNA | 27 | GTTWHA | 35 | AAAVSG |
| | | MG | | GTQYYAY | | PGYDS |
| | | | | AADSVKG | | |
| Ibx-11 | 20 | ASGLAFSI | 28 | VSVVDNT | 36 | YCGAGP |
| (flanking | | YGMGWF | | GSTTLYA | | KNYLGS |
| framework) | | | | DSVKGRF | | HEYAYW |
| | | | | | | G |
| Ibx-12 | 21 | ASGGTFTT | 29 | VSATTWS | 37 | YCSADP |
| (flanking | | YAMGWF | | TGTSYYA | | DGSDWA |
| framework) | | | | YAADSVK | | HSARQL |
| | | | | GRF | | RYWG |
| Ibx-13 | 22 | ASEDTFST | 30 | VAGITAN | 38 | SCGADT |
| (flanking | | YAIGWF | | GIATRYN | | RRFSLPS |
| framework) | | | | DSLKDRF | | TVHEIDY |
| | | | | | | WG |
| Ibx-14 | 23 | ASGRSLSS | 31 | VSGTTWH | 39 | YCAAAV |
| (flanking | | NAMGWF | | AGTQYYA | | SGPGYDS |
| framework) | | | | YAADSVK | | WG |
| | | | | GRF | | |
| Ibx-74 | | See Ibx-11 | | See Ibx-11 | | See Ibx-11 |
| (dimer of | | | | | | |
| Ibx-11) | | | | | | |
| Ibx-75 | | See Ibx-12 | | See Ibx-12 | | See Ibx-12 |
| (dimer of | | | | | | |
| Ibx-12) | | | | | | |
| Ibx-76 | | See Ibx-13 | | See Ibx-13 | | See Ibx-13 |
| (dimer of | | | | | | |
| Ibx-13) | | | | | | |
| Ibx-77 | | See Ibx-14 | | See Ibx-14 | | See Ibx-14 |
| (dimer of | | | | | | |
| Ibx-14) | | | | | | |

In some embodiments, an anti-CD147 antibody described herein can comprise a modification. In some embodiments, a modification comprises an amino acid substitution, deletion, and/or insertion. With regard to the modification, this may include any modification such as amino acid substitution, deletion and/or insertion at any location of a sequence. Hence, the modification may include a modification in sequence and/or length. In some embodiments, a sequence comprises a CDR region. A CDR may thus be modified to vary in length ranging from 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and up to about 35 amino acid residues. Similarly, any region of a CDR may be modified to include residue substitutions, deletions, and/or insertions ranging in length as previously described. In some embodiments, a modification can comprise from about 0-2 residue modifications. In some embodiments, a modification can comprise from about 1-3, 2-4, 0-4, or 1-5 residue modifications at any one of CDR1, CDR2, and/or CDR3. In some embodiments, any one of SEQ ID NO: 16-SEQ ID NO: 39 may have one or more modifications described herein.

In some embodiments, the anti-CD147 antibody or fragment thereof (e.g., VhH, CDR) is part of a chimeric antigen receptor (CAR). A CAR is a recombinant receptor composed of an extracellular antigen binding domain, a transmembrane domain, and an intracellular signaling domain. In some embodiments, the intracellular signaling domain may comprise signaling domains from an immune cell. In some embodiments, the immune cell is a mononuclear cell. In some embodiments, the mononuclear cell is a PBMC, PBL, lymphocyte, B cell, T cell, Natural Killer (NK) cell, monocyte, dendritic cell, eosinophil, neutrophil, basophil, or Antigen Presenting Cell (APC).

In some embodiments, a binding region of a CAR can comprise a CDR sequence disclosed herein. The binding region is normally fused to a transmembrane region, for example from CD8, which is also fused to an intracellular signaling domain. Suitable intracellular signaling domains can comprise an intracellular signal region sequence of CD3 zeta (CD3ζ), CD3 gamma, CD3 delta, CD3 epsilon, common FcR gamma (FCERIG), FcR beta (Fc Epsilon Rib), CD79a, CD79b, Fc gamma RIIa, DAP10 and DAP12 molecules, or a combination thereof. In some embodiments, the intracellular signal domain comprises a signaling domain of a CD3ζ molecule. In some embodiments, the intracellular signal domain also comprises a co-stimulatory signal domain, which may be selected from the following co-stimulatory signal molecules: CD27, CD28, 4-1BB, OX40, CD30, CD40, CD2, lymphocyte function-associated antigen-1 (LFA-1), LIGHT, NKG2C, B7-H3, PD-1, ICOS, CDS, ICAM-1, GITR, BAFFR, HVEM (LIGHTR), SLAMF7, CD7, NKp80 (KLRF1), CD160, CD19, CD4, CD8 alpha, CD8 beta, IL2R beta, IL2R gamma, IL7R alpha, ITGA4, VLAI, CD49a, ITGA4, IA4, CD49D, ITGA6, VLA-6, CD49f, ITGAD, CDIId, ITGAE, CD103, ITGAL, CDIIa, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMFI, CD150, IPO-3), BLAME (SLAMF8), SELPLG (CD162), LTBR, LAT, GADS, SLP-76, PAG/Cbp, NKp44, NKp30, NKp46, NKG2D, aligand specifically binding to CD83, or any combination thereof. In some embodiments, the costimulatory signal molecule is 4-1BB and/or CD28. In some embodiments, from 0-2 costimulatory signaling molecules are comprised in a CAR.

As used herein, “binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of kon/koff refers to the association rate constant of, e.g., an antibody to an antigen, and koff refers to the dissociation of, e.g., an antibody to an antigen. The kₒₙ and k_{off} can be determined by techniques known to one of ordinary skill in the art, such as BIACORE® or KinExA. In some embodiments, the antibody binds to CD147 with a KD of about 50 nM or lower. In some embodiments, the antibody binds to CD147 with a KD in a range of about 250 pM to about 1 pM. In some embodiments, the antibody binds to CD147 with a KD in a range of about 100 pM to about 1 pM. In some embodiments, antibody binds to CD147 with an IC50 of about 200 nM or lower. In some embodiments, the antibody binds to CD147 with an IC50 of about 100 nM to about 1 nM. In some embodiments, antibody binds to CD147 with an IC50 of about 10 nM to about 1 nM.

In some embodiment, the antibody or fragment thereof of the present disclosure binds to a CD147 on the surface of a cell. In some embodiments, the antibody binds to CD147 on the surface of a cancer cell. In some embodiments, the antibody binds to CD147 on the surface of a cancer cell that has not yet become metastatic. In some embodiments, the antibody binds to CD147 on the surface of a metastatic cancer cell.

In some embodiments, the antibody or fragment thereof of the present disclosure binds to CD147 on the surface of a virally-infected cell. In some embodiments, the antibody binds to CD147 on the surface of a cell that has not been infected by a virus. In some embodiments, binding of the antibody to the CD147 on the surface of the cell prevents viral infection. In some embodiments, the antibody interferes with the interaction of the virus and the CD147 on the surface of the cell. In some embodiments, the antibody interferes with the interaction of a spike protein and CD147. In some embodiments, the spike protein is a coronavirus spike protein. In some embodiments, the spike protein is a SARS-CoV2 spike protein. In some embodiments, the antibody interferes with the virus/CD147 interaction and inhibits viral invasion and/or dissemination among other cells.

In some embodiments, the antibody, antibody fragment, or modification thereof completely blocks the interaction of a viral protein and CD147. In some embodiments, the antibody, antibody fragment, or modification thereof decreases the interaction of a viral protein and CD147 compared to an untreated cell. In some embodiments, the antibody, antibody fragment, or modification thereof decreases the interaction of a viral protein and CD147 by about 99.9% to about 1% compared to an untreated cell. In some embodiments, the antibody, antibody fragment, or modification thereof decreases the interaction of a viral protein and CD147 by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to an untreated cell. In some embodiments, the antibody, antibody fragment, or modification thereof decreases the interaction of a viral protein and CD147 at about 10 minutes to about 1 day after administration compared to an untreated cell. In some embodiments, the antibody, antibody fragment, or modification thereof decreases the interaction of a viral protein and CD147 at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to an untreated cell.

In some embodiments, the antibody, antibody fragment, or modification thereof completely prevents viral invasion of a cell. In some embodiments, the antibody, antibody fragment, or modification thereof decreases viral invasion of a cell compared to an untreated cell. In some embodiments, the antibody, antibody fragment, or modification thereof decreases viral invasion of a cell by about 99.9% to about 1% compared to an untreated cell. In some embodiments, the antibody, antibody fragment, or modification thereof decreases viral invasion of a cell by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 710%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 310%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to an untreated cell. In some embodiments, the antibody decreases viral invasion of a cell at about 10 minutes to about 1 day after administration compared to an untreated cell. In some embodiments, the antibody decreases viral invasion of a cell at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to an untreated cell. In some embodiments, the cell is an epithelial cell. In some embodiments, the anti-CD147 antibody reduces or eliminates viral invasion of a cell by at least about 1-fold, 50-fold, 100-fold, 150-fold, 200-fold, 250-fold, 300-fold, 350-fold, 400-fold, 450-fold, 500-fold, 550-fold, 600-fold, 650-fold, 700-fold, 750-fold, 800-fold, 850-fold, 900-fold, 950-fold, 1000-fold, 1050-fold, 1100-fold, 1150-fold, 1200-fold, 1250-fold, 1300-fold, 1350-fold, 1400-fold, 1450-fold, 1500-fold, 1550-fold, 1600-fold, 1650-fold, 1700-fold, 1750-fold, 1800-fold, 1850-fold, 1900-fold, 1950-fold, or up to about 2000-fold, as determined by a viral infectivity assay described herein.

Viral invasion or infection can be determined using an in vitro assay. In some embodiments, a viral load can be quantified by counting viral RNA genomes by qRT-PCR and measuring the number of infectious units in tissue culture. The second approach comprises incubating susceptible mammalian cells with dilutions of a subject's sample to determine the amount of sample required to kill 50% of the cells. This value is used to back-calculate the infectious titer in the sample in units of “50% tissue culture infective dose” or TCID50 for example, by the Reed and Muench method.

In some embodiments, the antibody, antibody fragment, or modification thereof completely prevents viral dissemination among cells. In some embodiments, the antibody, antibody fragment, or modification thereof decreases viral dissemination among cells compared to that in an untreated subject or cell culture. In some embodiments, the antibody, antibody fragment, or modification thereof decreases viral dissemination among cells by about 99.9% to about 1% compared to that in an untreated subject or cell culture. In some embodiments, the antibody, antibody fragment, or modification thereof decreases viral dissemination among cells by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 210%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to that in an untreated subject or cell culture. In some embodiments, the antibody, antibody fragment, or modification thereof decreases viral dissemination among cells at about 10 minutes to about 1 day after administration compared to that in an untreated subject or cell culture. In some embodiments, the antibody, antibody fragment, or modification thereof decreases viral dissemination among cells at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to that in an untreated subject or cell culture.

In some embodiments, administration of an antibody, antibody fragment, or modification thereof of the present disclosure decreases inflammation in a subject. In some embodiments, administration of the antibody, antibody fragment, or modification thereof decreases inflammation compared to the levels of inflammation in a similar untreated subject or the same subject before administration of the antibody. In some embodiments, administration of the antibody, antibody fragment, or modification thereof decreases inflammation by about 99.9% to about 1% compared to the levels of inflammation in a similar untreated subject or the same subject before administration of the antibody. In some embodiments, administration of the antibody, antibody fragment, or modification thereof decreases inflammation by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels of inflammation in a similar untreated subject or the same subject before administration of the antibody. In some embodiments, administration of the antibody, antibody fragment, or modification thereof decreases inflammation at about 10 minutes to about 1 day after administration compared to the levels of inflammation in a similar untreated subject or the same subject before administration of the antibody. In some embodiments, administration of the antibody, antibody fragment, or modification thereof decreases inflammation at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels of inflammation in a similar untreated subject or the same subject before administration of the antibody.

In some embodiments, the inflammation results in inflammatory lung damage. The decrease in inflammatory lung damage may be measured by any means known in the art. For example, inflammatory lung damage and/or the symptoms thereof may be measured by assessing changes in oxygen levels in the patient, exhaled nitric oxide, respiratory rate, or spirometry measurements.

In some embodiments, administration of an antibody, antibody fragment, or modification thereof of the present disclosure decreases proteinase activity in a subject. In some embodiments, the proteinase is a metalloproteinase. In some embodiments, administration of the antibody, antibody fragment, or modification thereof decreases proteinase activity compared to the levels in a similar untreated subject or the same subject before administration of the antibody. In some embodiments, administration of the antibody, antibody fragment, or modification thereof decreases proteinase activity by about 99.9% to about 1% compared to the levels in a similar untreated subject or the same subject before administration of the antibody. In some embodiments, administration of the antibody, antibody fragment, or modification thereof decreases proteinase activity by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases proteinase activity at about 10 minutes to about 1 day after administration compared to the levels of a similar untreated subject or the same subject before administration of the antibody. In some embodiments, administration of the antibody, antibody fragment, or modification thereof decreases proteinase activity at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels of in a similar untreated subject or the same subject before administration of the antibody. In some embodiments, the proteinase is produced and/or secreted by a cell expressing CD147. In some embodiments, the proteinase is produced and/or secreted by a cancer cell. In some embodiments, the proteinase is produced and/or secreted by a cancer cell expressing CD147.

In some embodiments, administration of an antibody, antibody fragment, or modification thereof of the present disclosure decreases matrix degradation in a subject. In some embodiments, administration of the antibody, antibody fragment, or modification thereof decreases matrix degradation compared to the levels in a similar untreated subject or the same subject before administration of the antibody. In some embodiments, administration of the antibody, antibody fragment, or modification thereof decreases matrix degradation by about 99.9% to about 1% compared to the levels in a similar untreated subject or the same subject before administration of the antibody. In some embodiments, administration of the antibody, antibody fragment, or modification thereof decreases matrix degradation by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 210%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases matrix degradation at about 10 minutes to about 1 day after administration compared to the levels in a similar untreated subject or the same subject before administration of the antibody. In some embodiments, administration of the antibody, antibody fragment, or modification thereof decreases matrix degradation at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels in a similar untreated subject or the same subject before administration of the antibody.

In some embodiments, administration of an antibody, antibody fragment, or modification thereof of the present disclosure decreases cell invasion in a subject. In some embodiments, administration of the antibody, antibody fragment, or modification thereof decreases cell invasion compared to the levels in a similar untreated subject or the same subject before administration of the antibody. In some embodiments, administration of the antibody, antibody fragment, or modification thereof decreases cell invasion by about 99.9% to about 1% compared to the levels in a similar untreated subject or the same subject before administration of the antibody. In some embodiments, administration of the antibody, antibody fragment, or modification thereof decreases cell invasion by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases cell invasion at about 10 minutes to about 1 day after administration compared to the levels in a similar untreated subject or the same subject before administration of the antibody. In some embodiments, administration of the antibody, antibody fragment, or modification thereof decreases cell invasion at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels in a similar untreated subject or the same subject before administration of the antibody. In some embodiments, the cell is a tumor cell.

In some embodiments, administration of an antibody, antibody fragment, or modification thereof of the present disclosure decreases tumor cell metastasis in a subject. In some embodiments, administration of the antibody, antibody fragment, or modification thereof decreases tumor cell metastasis compared to the levels in a similar untreated subject or the same subject before administration of the antibody. In some embodiments, administration of the antibody, antibody fragment, or modification thereof decreases tumor cell metastasis by about 99.9% to about 1% compared to the levels in a similar untreated subject or the same subject before administration of the antibody. In some embodiments, administration of the antibody, antibody fragment, or modification thereof decreases tumor cell metastasis by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 110, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases tumor cell metastasis at about 10 minutes to about 1 day after administration compared to the levels in a similar untreated subject or the same subject before administration of the antibody. In some embodiments, administration of the antibody, antibody fragment, or modification thereof decreases tumor cell metastasis at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels in a similar untreated subject or the same subject before administration of the antibody.

In some embodiments, administration of an antibody, antibody fragment, or modification thereof of the present disclosure decreases tumor cell viability in a subject. In some embodiments, administration of the antibody, antibody fragment, or modification thereof decreases tumor cell viability compared to the levels in a similar untreated subject or the same subject before administration of the antibody. In some embodiments, administration of the antibody, antibody fragment, or modification thereof decreases tumor cell viability by about 99.9% to about 1% compared to the levels in a similar untreated subject or the same subject before administration of the antibody. In some embodiments, administration of the antibody, antibody fragment, or modification thereof decreases tumor cell viability by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 210%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases tumor cell viability at about 10 minutes to about 1 day after administration compared to the levels in a similar untreated subject or the same subject before administration of the antibody. In some embodiments, administration of the antibody, antibody fragment, or modification thereof decreases tumor cell viability at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels in a similar untreated subject or the same subject before administration of the antibody.

In some embodiments, administration of an antibody, antibody fragment, or modification thereof of the present disclosure decreases angiogenesis in a subject. In some embodiments, administration of the antibody, antibody fragment, or modification thereof decreases angiogenesis compared to the levels in a similar untreated subject or the same subject before administration of the antibody. In some embodiments, administration of the antibody, antibody fragment, or modification thereof decreases angiogenesis by about 99.9% to about 1% compared to the levels in a similar untreated subject or the same subject before administration of the antibody. In some embodiments, administration of the antibody, antibody fragment, or modification thereof decreases angiogenesis by about 99.9%, about 99.8%, about 99.7%, about 99.6%, about 99.5%, about 99.4%, about 99.3%, about 99.2%, about 99.1%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% compared to the levels in a similar untreated subject or the same subject before administration of the antibody. In some embodiments, administration of the antibody, antibody fragment, or modification thereof decreases angiogenesis at about 10 minutes to about 1 day after administration compared to the levels in a similar untreated subject or the same subject before administration of the antibody. In some embodiments, administration of the antibody, antibody fragment, or modification thereof decreases angiogenesis at about 5 minutes, about 10 minutes, about 20 minutes, about 30 minutes, about 40 minutes, about 50 minutes, about 60 minutes, about 70 minutes, about 80 minutes, about 90 minutes, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours after administration compared to the levels in a similar untreated subject or the same subject before administration of the antibody. In some embodiments, the subject has cancer. In some embodiments, the angiogenesis is tumor-specific angiogenesis.

### Antibody-Drug Conjugate (ADC)

In some embodiments, any of the provided anti-CD147 antibodies may be fused to a drug as part of an ADC. In some embodiments, an antibody comprises any of the aforementioned constructs described in Table 1 or Table 2. Antibody-Drug Conjugates (ADCs) can be built by attaching a small molecule drug or another therapeutic agent to an antibody, with either a permanent or a labile linker. The antibody targets a specific antigen typically found on target cells. Once it binds to the cell, it triggers internalization of the antibody, together with the drug. This delivers drugs with a very high specificity to the diseased or target cells, maximizing their efficacy and minimizing systemic exposure, with the associated risk of side effects. In some embodiments, a linker is utilized to connect an antibody with a drug. The linker is normally stabilized to avoid the release of the drug around off-target tissue, and the linker may maintain the conjugate in an inactive, nontoxic state while bound to the antibody. In some embodiments, the linker should possess the property of unleashing the drug upon internalization of the ADC into a target cell. In some embodiments, any of the linkers previously described can be utilized in an ADC construct. In some embodiments, a drug that is part of an ADC is selected from the group consisting of an: anti-cancer drug, anti-malaria drug, anti-viral drug, an anti-inflammatory drug, and a toxin.

In some embodiments, the drug is an anti-viral drug. In some embodiments, the anti-viral drug is an anti-SARS-CoV-2 drug. In some embodiments, the anti-SARS-CoV-2 drug comprises a monoclonal antibody including but not limited to: bamlanivimab, etesevimab, casirivimab, imdevimab, and sotrovimab. In some embodiments, an anti-SARS-CoV-2 drug is not an antibody. In some embodiments, a drug is an anti-cancer drug. In some embodiments, an anti-cancer drug comprises an immunotherapy. In some embodiments, the immunotherapy is selected from the group consisting of an antibody, checkpoint inhibitor, cell therapy, cytokine, oncolytic virus, and vaccine. In some embodiments, an anti-cancer drug comprises a cytotoxic agent. In some embodiments, the immunotherapy comprises the antibody. Antibodies that are contemplated include gemtuzumab, inotuzumab, anti-B4-bR, denileukin, and combinations thereof. In some embodiments, the drug comprises a toxin. Suitable toxins comprise Pseudomonas exotoxin and/or diphtheria toxin. In some embodiments, an ADC comprises the anti-CD147 antibody of SEQ ID NO: 14. In some embodiments, an ADC comprises the anti-CD147 antibody of SEQ ID NO: 12. In some embodiments, the drug comprises a growth factor. Exemplary growth factors include but are not limited to: IL-2, IL-13, TGFα, GM-CSF, and combinations thereof.

### Methods of Treatment

In some embodiments, the present disclosure provides methods of treating, ameliorating, or preventing a disorder associated with CD147 expression and/or a symptom thereof.

In some embodiments, the present disclosure provides methods of treating, ameliorating, or preventing cancer comprising administering pharmaceutical compositions of an anti-CD147 antibody, antibody fragment, or modification thereof of the disclosure to a subject in need. In some embodiments, administration of an anti-CD147 antibody, antibody fragment, or modification thereof of the disclosure prevents or delays the growth of a tumor or proliferation of cancer cells in the subject. In some embodiments, administration of an anti-CD147 antibody, antibody fragment, or modification thereof of the disclosure prevents or delays the progression of cancer cells in the subject. In some embodiments, administration of an anti-CD147 antibody, antibody fragment, or modification thereof of the disclosure prevents or delays cancer cell metastasis in the subject. In some embodiments, administration of an anti-CD147 antibody, antibody fragment, or modification thereof of the disclosure prevents or delays angiogenesis in the subject. In some embodiments, administration of an anti-CD147 antibody, antibody fragment, or modification thereof of the disclosure prevents or delays cell invasion in the subject. In some embodiments, the cancer includes, but is not limited to, breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

In some embodiments, the present disclosure provides methods of treating, ameliorating, or preventing an inflammatory or autoimmune disorder comprising administering pharmaceutical compositions of an anti-CD147 antibody, antibody fragment, or modification thereof of the disclosure to a subject in need. In some embodiments, the autoimmune or inflammatory disorder includes, but is not limited to, rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

In some embodiments, the present disclosure provides a method of treating or preventing viral infection symptoms, and/or related conditions comprising administering a pharmaceutical composition comprising an anti-CD147 antibody, antibody fragment, or modification thereof of the disclosure to a subject in need. In some embodiments, the viral infection is caused by a virus including, but not limited to, measles, coronavirus, SARS, MERS, SARS-CoV-2, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis. In some embodiments, the viral infection is a coronavirus infection. In some embodiments, the coronavirus infection is a SARS-CoV-2 infection. Any strain of SARS-CoV-2 can be treated with the antibody of the disclosure including but not limited to: alpha, beta, gamma, delta, epsilon, zeta, eta, theta, iota, kappa, lambda, mu, nu, xi, omicron, pi, rho, sigma, tau, upsilon, phi, chi, psi, or omega. In some embodiments, the SARS-CoV-2 strain is one or more of: Alpha, Beta, Gamma, Delta, and/or Omicron.

In some embodiments, the symptom and/or related condition is respiratory distress and/or failure. In some embodiments, respiratory failure is defined based on resource utilization requiring at least one of the following: endotracheal intubation and mechanical ventilation, oxygen delivered by high-flow nasal cannula; heated, humidified, oxygen delivered via reinforced nasal cannula at flow rates >20 L/min with fraction of delivered oxygen ≥0.5, noninvasive positive pressure ventilation, extracorporeal membrane oxygenation (ECMO), and/or clinical diagnosis of respiratory failure (e.g. clinical need for one of the preceding therapies, but preceding therapies not able to be administered in settings of resource limitation).

### Pharmaceutical Compositions and Administration

The present disclosure provides pharmaceutical compositions including an anti-CD147 antibody, antibody fragment, or modification thereof of the disclosure with one or more pharmaceutically acceptable excipients and/or diluents. For example, such excipients include salts, and other excipients that may act to stabilize hydrogen bonding. Any appropriate excipient known in the art may be used. Exemplary excipients include, but are not limited to, amino acids such as histidine, glycine, or arginine; glycerol; sugars, such as sucrose; surface active agents such as polysorbate 20 and polysorbate 80; citric acid; sodium citrate; antioxidants; salts including alkaline earth metal salts such as sodium, potassium, and calcium; counter ions such as chloride and phosphate; preservatives; sugar alcohols (e.g., mannitol, sorbitol); and buffering agents. Exemplary salts include sodium chloride, potassium chloride, magnesium chloride, calcium chloride, sodium phosphate dibasic, sodium phosphate monobasic, potassium phosphate monobasic, and potassium phosphate dibasic. In certain embodiments, the pharmaceutical compositions disclosed herein have enhanced efficacy, bioavailability, therapeutic half-life, persistence, degradation resistance, etc.

The pharmaceutical formulation can be stored frozen, refrigerated or at room temperature. The storage condition may be below freezing, such as lower than about −10° C., or lower than about −20° C., or lower than about −40° C., or lower than about −70° C. In some embodiments, the pharmaceutical formulation is stored at 2°-8° C. For example, the pharmaceutical formulation may be isotonic with blood or have an ionic strength that mimics physiological conditions. For example, the formulation may have an ionic strength of at least that of 25 mM Sodium Chloride, or at least that of 30 mM Sodium chloride, or at least that of 40 mM Sodium Chloride, or at least that of 50 mM Sodium Chloride, or at least that of 75 mM Sodium Chloride, or at least that of 100 mM Sodium Chloride, or at least that of 150 mM Sodium Chloride. In certain embodiments, the formulation has an ionic strength equivalent to that of 0.9% saline (154 mM sodium chloride).

In some embodiments, the pharmaceutical formulation is formulated at physiological pH. In some embodiments, the pharmaceutical formulation is formulated at a pH in the range of about 5.5 to about 8.5. In some embodiments, the pharmaceutical formulation is formulated at a pH in the range of about 6.0 to about 8.0. In some embodiments, the pharmaceutical formulation is formulated at a pH in the range of about 6.5 to about 7.5. In some embodiments, the pharmaceutical formulation is formulated at a pH of 7.5. In some embodiments, pharmaceutical formulations with a lower pH demonstrate improved formulation stability compared to formulations at a higher pH. In some embodiments, pharmaceutical formulations with a higher pH demonstrate improved formulation stability compared to formulations at a lower pH.

In some embodiments, the pharmaceutical formulation is stable at storage conditions. Stability can be measured using any appropriate means in the art. Generally, a stable formulation is one that shows less than a 5% increase in degradation products or impurities. In some embodiments, the formulation is stable for at least about 1 month, at least about 2 months, at least about 3 months, at least about 4 months, at least about 5 months, at least about 6 months, at least about one year, or at least about 2 years or more at the storage conditions. In some embodiments, the formulation is stable for at least about 1 month, at least about 2 months, at least about 3 months, at least about 4 months, at least about 5 months, at least about 6 months, or at least about one year or more at 25° C.

In another embodiment, the disclosure provides a method for delivering a pharmaceutical formulation disclosed herein. The method comprises administering the pharmaceutical formulation described herein to a subject in need, wherein the pharmaceutical formulation is administered from about 1 to about 31 times per month. In some embodiments, the pharmaceutical formulation is administered about 1 time, about 2 times, about 3 times, and/or about 4 times per month. In some embodiments, the pharmaceutical composition is administered daily. In some embodiments, the pharmaceutical is administered weekly. In some embodiments, the pharmaceutical composition is administered weekly. In some embodiments, the pharmaceutical composition is administered from one to three times weekly. In some embodiments, the pharmaceutical composition is administered once every two weeks. In some embodiments, the pharmaceutical composition is administered from one to two times a month. In some embodiments, the pharmaceutical composition is administered about 1 time per month. In some embodiments, the pharmaceutical composition is administered about once every 2 months, about once every 3 months, about once every 4 months, about once every 5 months, and/or about once every 6 months.

In some embodiments, the pharmaceutical compositions disclosed herein are administered until at least one or more symptoms of the disorder being treated is reduced and/or improve. In some embodiments, the pharmaceutical compositions disclosed herein are administered until at least one or more symptoms is ameliorated. In some embodiments, the pharmaceutical compositions disclosed herein are administered until at least one or more symptoms thereof is delayed. In some embodiments, the pharmaceutical compositions disclosed herein are administered until the disorder is cured.

In some embodiments, the pharmaceutical compositions disclosed herein are administered before the patient begins to exhibit one or symptoms. In some embodiments, the pharmaceutical compositions disclosed herein are administered at the onset of symptoms.

The pharmaceutical composition may be administered by any known route, such as for example, orally, intravenously, intramuscularly, nasally, subcutaneously, parenterally, via inhalation, or intradermally. In some embodiments, the formulation is generally for subcutaneous administration. In some embodiments, the formulation is generally for intravenous administration.

While one of skill in the art can determine the desirable dose in each case, a suitable dose of the pharmaceutical composition for achievement of therapeutic benefit, may, for example, be in a range of about 1 microgram (μg) to about 100 milligrams (mg) per kilogram body weight of the recipient, preferably in a range of about 10 μg to about 50 mg per kilogram body weight and most preferably in a range of about 100 μg to about 10 mg per kilogram body weight. In some embodiments, the pharmaceutical composition is administered at a low dose. In some embodiments, the pharmaceutical composition is administered at a dose between 0.1 mg per kilogram per body weight to about 9 mg per kilogram per body weight. In some embodiments, the pharmaceutical composition is administered at about 0.05 mg per kilogram body weight, about 0.1 mg per kilogram body weight, about 0.2 mg per kilogram body weight, about 0.4 mg per kilogram body weight, about 0.5 mg per kilogram body weight, about 0.8 mg per kilogram body weight, about 1 mg per kilogram body weight, about 1.2 mg per kilogram body weight, about 2 mg per kilogram body weight, about 3 mg per kilogram body weight, and/or about 9 mg per kilogram body weight. The desired dose may be administered weekly.

A suitable dose of the pharmaceutical composition for achievement of therapeutic benefit, may, for example, be in a range of about 1 microgram (μg) to about 10 milligrams (mg) per kilogram body weight of the recipient, preferably in a range of about 10 μg to about 5 mg per kilogram body weight, and most preferably in a range of about 100 μg to about 2 mg per kilogram body weight. In some embodiments, the pharmaceutical composition is administered at a low dose. In some embodiments, the pharmaceutical composition is administered at a dose between 0.1 mg per kilogram per body weight to about 9 mg per kilogram per body weight. In some embodiments, the pharmaceutical composition is administered at about 0.05 mg per kilogram body weight, about 0.1 mg per kilogram body weight, about 0.2 mg per kilogram body weight, about 0.4 mg per kilogram body weight, about 0.5 mg per kilogram body weight, about 0.8 mg per kilogram body weight, about 1 mg per kilogram body weight, about 1.2 mg per kilogram body weight, about 2 mg per kilogram body weight, about 3 mg per kilogram body weight, and/or about 9 mg per kilogram body weight. The desired dose may be presented as one dose, or two or more sub-doses administered at appropriate intervals. These sub-doses can be administered in unit dosage forms, for example, containing from about 10 μg to about 1000 mg, preferably from about 50 μg to about 500 mg, and most preferably from about 50 μg to about 250 mg of active ingredient per unit dosage form. Alternatively, if the condition of the recipient so requires, the doses may be administered as a continuous infusion.

In some embodiments, the pharmaceutical composition is administered in a fixed dose, regardless of the weight of the patient at a dose of about 1 mg to about 1 g or more. In some embodiments, the pharmaceutical composition is administered at a dose of about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 600 mg, about 700 mg, about 800 mg, about 900 mg, about 1 g or more. In some embodiments, the pharmaceutical composition is administered at a dose of about 10 mg, 40 mg, or 100 mg.

The pharmaceutical composition may be administered for between about one day to about one year or more. In some embodiments, the pharmaceutical composition is administered for one day, two days, three days, four days, five days, six days, seven days, two weeks, three weeks, four weeks, one month, two months, three months, four months, five months, six months, seven months, eight months, nine months, ten months, eleven months, twelve months or more.

In certain embodiments, the subject is a human, but in other embodiments may be a non-human mammal, such as a domesticated pet (e.g., dog or cat), or livestock or farm animal (e.g., horse, cow, sheep, or pig).

### Patient Population

Any appropriate subject may be administered the pharmaceutical compositions of the present disclosure. In some embodiments, the subject is at risk of viral infection, development of cancer, or development of an autoimmune or inflammatory disorder. In some embodiments, the patient is at risk of developing harmful or life-threatening effects of a disorder described herein. In some embodiments, the subject is experiencing harmful or life-threatening effects of a disorder described herein.

In some embodiments, the subject is infected with, or presumed to be infected with, SARS-CoV-2. In some embodiments, the subject has COVID-19, ARDS, and/or is experiencing symptoms associated with COVID-19 or ARDS. In some embodiments, the subject is hospitalized. In some embodiments, the patient has a mild case of COVID-19, ARDS, and/or is experiencing mild symptoms associated with COVID-19 or ARDS. In some embodiments, the subject has a moderate case of COVID-19, ARDS, and/or is experiencing moderate symptoms associated with COVID-19 or ARDS. In some embodiments, the subject has a severe case of COVID-19, ARDS, and/or is experiencing severe symptoms associated with COVID-19 (e.g., rapid clinical deterioration, ARDS, and/or death). In some embodiments, the patient requires assistance breathing. In some embodiments, the patient is receiving supplemental oxygen. In some embodiments, the patient is receiving supplemental oxygen by face mask or nasal cannula with prongs. In some embodiments, the patient requires a ventilator to breathe. In some embodiments, the patient exhibits low oxygen saturation levels. In some embodiments, the patient exhibits an increased respiratory rate (e.g., greater than 24 breaths/minute). In some embodiments, the patient exhibits an accompanying fever (e.g., temperature greater than 100.4° F. or 38° C.). In some embodiments, the patient is at risk of progressing to more severe COVID-19, ARDS, or symptoms thereof, and the pharmaceutical composition of the present disclosure is administered before symptoms worsen.

In some embodiments, the patient is determined as having mild COVID-19 by 1) positive testing by standard RT-PCR assay or the equivalent; 2) symptoms of mild illness with COVID-19 that could include fever, cough, sore throat, malaise, headache, muscle pain, gastrointestinal symptoms, without shortness of breath of dyspnea; and/or 3) no clinical signs indicative of moderate, severe, or critical COVID-19.

In some embodiments, the patient is determined as having moderate COVID-19 by 1) positive testing by standard RT-PCR assay or the equivalent; 2) symptoms of moderate illness with COVID-19, which can include any symptom of mild illness or shortness of breath with exertion; 3) clinical signs suggestive of moderate illness with COVID-19, such as respiratory rate ≥20 breaths per minute, saturation of oxygen (SpO₂)>93% on room air at sea level, heart rate ≥90 beats per minute; and/or 4) no clinical signs indicative of severe or critical illness.

In some embodiments, the patient is determined as having severe COVID-19 by 1) positive testing by standard RT-PCR assay or the equivalent; 2) symptoms suggestive of severe systemic illness with COVID-19, which can include any symptom of moderate illness or shortness of breath at rest, or respiratory distress; 3) clinical signs indicative of severe systemic illness with COVID-19, such as respiratory rate ≥30 per minute, heart rate ≥125 per minute, SpO₂≤93% on room air at sea level, or PaO₂/FiO₂<300 and/or 4) no criteria for critical seventy.

In some embodiments, the patient is determined as having severe COVID-19 by 1) positive testing by standard RT-PCR assay or the equivalent; 2) evidence of critical illness, defined by at least one of the following: a) respiratory failure defined based on resource utilization requiring at least one of the following: endotracheal intubation and mechanical ventilation, oxygen delivered by high-flow nasal cannula; heated, humidified, oxygen delivered via reinforced nasal cannula at flow rates >20 L/min with fraction of delivered oxygen ≥0.5, noninvasive positive pressure ventilation, extracorporeal membrane oxygenation (ECMO), and/or clinical diagnosis of respiratory failure (e.g. clinical need for one of the preceding therapies, but preceding therapies not able to be administered in settings of resource limitation); b) shock (e.g. defined by systolic blood pressure <90 mmHg, or diastolic blood pressure <60 mmHg, or requiring vasopressors); and/or c) multi-organ dysfunction/failure.

In some embodiments, the patient is an infant (e.g., 0 to 2 years old inclusive). In some embodiments, the patient is a pediatric patient (e.g., 2 to 18 years old inclusive). In some embodiments the patient is between the ages of 18 and 100. In some embodiments, the patient is between the ages of 18 and 85. In some embodiments, the patient is between the ages of 50 and 100. In some embodiments, the patient is older than 65 years old. In some embodiments, the patient is 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old or older. In some embodiments, the patient who is older than 50 years old is considered high risk of developing severe disease (e.g., COVID-19 or ARDS). In some embodiments, the patient who is older than 65 years old is considered high risk of developing severe disease (e.g., COVID-19 or ARDS).

In some embodiments, a patient who possesses one or more comorbidities is considered high risk for developing severe disease (e.g., COVID-19 or ARDS). In some embodiments, the comorbidity includes, but is not limited to, obesity, hypertension, diabetes, an autoimmune disorder (e.g., rheumatoid arthritis), heart disease, heart failure, atherosclerosis, cancer (e.g., lung cancer), exposure to lung-damaging agents, liver disease, alcoholism, other pulmonary infection, and chronic kidney disease. In some embodiments, the patient at risk presents with elevated markers of cardiac injury or dysfunction (e.g., hsTnI, NT-proBNP). In some embodiments, race is a factor in a patient being considered at high risk of developing severe disease (e.g., COVID-19 or ARDS). In some embodiments, socioeconomic status is a factor in a patient being considered at high risk of developing severe disease (e.g., COVID-19 or ARDS).

In some embodiments, while being administered with a pharmaceutical composition of the present disclosure, the patient continues to receive standard of care for any comorbidities.

### Combination Therapies

The pharmaceutical compositions disclosed herein may be administered with various therapies used to treat, prevent, delay, or ameliorate cancers, viral infection, inflammation, autoimmune disorders, inflammatory lung damage, COVID-19, and/or ARDS. In some embodiments, the pharmaceutical composition is administered concomitantly with standard of care medications. The one or more therapeutic agents may be any compound, molecule, or substance that exerts therapeutic effect to a subject in need thereof.

In some embodiments, the pharmaceutical compositions disclosed herein are administered with therapeutic agents including, but not limited to, antiviral agents, anti-malarial agents, agents that protect epithelial cells, defibrotide, convalescent plasma, chloroquine, hydroxychloroquine, remdesivir, desferal, favipiravir, corticosteroids, clevudine, anti-inflammatory agents, anti-oxidant agents, dapagliflozin, IFX-1, ruxolitinib, baricitinib, interferon beta 1a, azithromycin, tocilizumab, acalabrutinib, umifenovir, ciclesonide, sarilumab, anti-interleukin agents, and telmisartan.

In some embodiments, a subject has previously been treated or undergoes combination therapy with an anti-SARS-CoV-2 therapy or COVID-19 therapy. Suitable therapies comprise: monoclonal antibodies (e.g., bamlanivimab, evusheld, sotrovimab, Regen-Cov, and etesevimab), Remdesivir, mechanical ventilation, oxygen therapy, and combinations thereof. In some embodiments, a subject in need thereof is recalcitrant to an anti-SARS-CoV-2 therapy or COVID-19 therapy for example those suitable therapies provided herein. In some embodiments, a subject in need thereof that is recalcitrant to an anti-SARS-CoV-2 therapy is treated with an antibody of the disclosure.

In some embodiments, the pharmaceutical compositions disclosed herein are administered with one or more chemotherapeutic agents, including but not limited to, methotrexate, daunomycin, mitomycin, cisplatin, vincristine, epirubicin, fluorouracil, verapamil, cyclophosphamide, cytosine arabinoside, aminopterin, bleomycin, mitomycin C, democolcine, etoposide, mithramycin, chlorambucil, melphalan, daunorubicin, doxorubicin, tamoxifen, paclitaxel, vinblastine, camptothecin, actinomycin D, cytarabine, and combrestatin.

The one or more therapeutic agents may be “co-administered”, i.e., administered together in a coordinated fashion to a subject, either as separate pharmaceutical compositions or admixed in a single pharmaceutical composition. By “co-administered”, the one or more therapeutic agents may also be administered simultaneously with the present pharmaceutical compositions, or be administered separately, including at different times and with different frequencies. The one or more therapeutic agents may be administered by any known route, such as orally, intravenously, intramuscularly, nasally, via aerosol, subcutaneously, intra-vaginally, intra-rectally, and the like; and the therapeutic agent may also be administered by any conventional route. In some embodiments, the pharmaceutical composition is administered subcutaneously.

When two or more therapeutic agents are used in combination, the dosage of each therapeutic agent is commonly identical to the dosage of the agent when used independently. However, when a therapeutic agent interferes with the metabolism of others, the dosage of each therapeutic agent is properly adjusted. Alternatively, where the two or more therapeutic agents show synergistic effects, the dose of one or more may be reduced. Each therapeutic agent may be administered simultaneously or separately in an appropriate time interval.

### Definitions

As used in this disclosure, the singular forms “a,” “an” and “the” include plural referents unless the context clearly dictates otherwise.

As used in this disclosure, the term “or” is understood to be inclusive and covers both “or” and “and”.

As used in this disclosure, any concentration range, percentage range, ratio range or integer range is to be understood to be inclusive of the value of any integer within the recited range and, when appropriate, fractions thereof (such as one-tenth and one-hundredth of an integer), unless otherwise indicated. Units, prefixes, and symbols used herein are provided using their Systeme International de Unites (SI) accepted form. Numeric ranges are inclusive of the numbers defining the range.

The terms “peptide,” “polypeptide,” and “protein” are used interchangeably, and refer to a compound comprising amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

To calculate percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm). In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89: 10915-10919 for the BLOSUM 62 comparison matrix) is also used by the algorithm.

### Examples of Non-Limiting Embodiments of the Disclosure

Embodiments of the present subject matter disclosed herein may be beneficial alone or in combination with one or more other embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Embodiments

Embodiment 1. An anti-CD147 antibody comprising a binding domain that comprises at least one CDR comprising at least 70% identity to an amino acid sequence selected from SEQ ID NOs: 16-39.

Embodiment 2. The anti-CD147 antibody of embodiment 1, wherein the anti-CD147 antibody comprises two CDRs, wherein each of the two CDRs independently comprise at least 70% identity to an amino acid sequence selected from SEQ ID NOs: 16-39.

Embodiment 3. The anti-CD147 antibody of embodiments 1 or 2, wherein the anti-CD147 antibody comprises three CDRs, wherein each of the three CDRs independently comprise at least 70% identity to an amino acid sequence selected from SEQ ID NOs: 16-39.

Embodiment 4. The anti-CD147 antibody of any one of embodiments 1-3, wherein the anti-CD147 antibody comprises an amino acid sequence with at least 70% identity to a sequence selected from SEQ ID NOs: 4-15.

Embodiment 5. The anti-CD147 antibody of any one of embodiments 1-4, wherein the anti-CD147 antibody comprises an amino acid sequence selected from SEQ ID NOs: 4-15.

Embodiment 6. The anti-CD147 antibody of any one of embodiments 1-5, wherein the antibody is a full-length antibody, a monospecific antibody, a bispecific antibody, a trispecific antibody, an antigen-binding region, heavy chain, light chain, VhH, Vh, a CDR, a variable domain, scFv, Fc, Fv, Fab, F(ab)2, IgG, reduced IgG (rIgG), monospecific Fab2, bispecific Fab2, trispecific Fab3, diabody, bispecific diabody, trispecific triabody, minibody, nanobody, IgNAR, V-NAR, HcIgG, or a combination thereof.

Embodiment 7. The anti-CD147 antibody of embodiment 6, wherein the antibody is the VhH.

Embodiment 8. The anti-CD147 antibody of any one of embodiments 1-7, wherein a binding region of a Chimeric Antigen Receptor (CAR) comprises at least one CDR comprising at least 70% identity to an amino acid sequence selected from SEQ ID NOs: 16-39.

Embodiment 9. The anti-CD147 antibody of embodiment 8, wherein the CAR is expressed on an immune cell.

Embodiment 10. The anti-CD147 antibody of embodiment 9, wherein the immune cell is a peripheral blood mononuclear cell (PBMC), a lymphocyte, a T cell, or a NK cell.

Embodiment 11. The anti-CD147 antibody of any one of embodiments 1-10, wherein the anti-CD147 antibody binds to a CD147 or fragment thereof expressed on the surface of a cell.

Embodiment 12. The anti-CD147 antibody of embodiment 11, wherein the cell is an epithelial cell, an endothelial cell, or neuronal cell.

Embodiment 13. The anti-CD147 antibody of any one of embodiments 1-12, wherein the anti-CD147 antibody reduces or eliminates interaction between a virus and CD147.

Embodiment 14. The anti-CD147 antibody of embodiment 13, wherein the interaction comprises binding of the CD147 by the virus.

Embodiment 15. The anti-CD147 antibody of any one of embodiments 1-14, wherein the anti-CD147 antibody reduces or eliminates a viral spike protein from binding to CD147.

Embodiment 16. The anti-CD147 antibody of any one of embodiments 1-15, wherein the anti-CD147 antibody reduces or eliminates viral invasion of a cell.

Embodiment 17. The anti-CD147 antibody of any one of embodiments 1-16, wherein the anti-CD147 antibody reduces or eliminates viral invasion of a cell by at least about 1-fold as determined by a viral infectivity assay.

Embodiment 18. The anti-CD147 antibody of any one of embodiments 13-17, wherein the virus is selected from the group consisting of: measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis.

Embodiment 19. The anti-CD147 antibody of embodiment 18, wherein the virus is the coronavirus.

Embodiment 20. The anti-CD147 antibody of embodiment 19, wherein the coronavirus is SARS-CoV-2.

Embodiment 21. The anti-CD147 antibody of any one of embodiments 11-20, wherein the cell is a cancer cell.

Embodiment 22. The anti-CD147 antibody of any one of embodiments 11-20, wherein the cell is a tumor cell.

Embodiment 23. The anti-CD147 antibody of embodiment 21, wherein the cancer cell is from a hematological cancer.

Embodiment 24. The anti-CD147 antibody of embodiment 22, wherein the tumor cell is from a cancer selected from the group consisting of: breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

Embodiment 25. The anti-CD147 antibody of any one of embodiments 1-24, wherein the anti-CD147 antibody reduces or eliminates tumor cell proliferation, metastasis, secretion of matrix metalloproteinases, degradation of a tumor matrix, tumor cell invasion, and/or angiogenesis.

Embodiment 26. The anti-CD147 antibody of any one of embodiments 1-24, wherein the anti-CD147 antibody reduces or eliminates inflammation.

Embodiment 27. The anti-CD147 antibody of any one of embodiments 1-24, wherein the anti-CD147 antibody is effective in reducing or eliminating an inflammatory or autoimmune disorder.

Embodiment 28. The anti-CD147 antibody of embodiment 27, wherein the inflammatory or autoimmune disease is selected from the group consisting of: rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

Embodiment 29. The anti-CD147 antibody of any one of embodiments 1-28, wherein the anti-CD147 antibody is humanized.

Embodiment 30. A method of preventing, treating, or ameliorating a disorder associated with CD147 expression in a subject in need thereof comprising administering the anti-CD147 antibody of any one of embodiments 1-29.

Embodiment 31. A pharmaceutical composition comprising an effective amount of the anti-CD147 antibody of any one of embodiments 1-29.

Embodiment 32. A cell comprising a sequence encoding the anti-CD147 antibody of any one of embodiments 1-29.

Embodiment 33. The anti-CD147 antibody of any one of embodiments 1-29, wherein the anti-CD147 antibody binds to a cell thereby preventing, inhibiting, or decreasing invasion of a Plasmodium parasite into the cell.

Embodiment 34. The anti-CD147 antibody of embodiment 33, wherein the invasion of the Plasmodium parasite into the cell is by at least about 1-fold.

Embodiment 35. The anti-CD147 antibody of embodiment 34, wherein the cell is a red blood cell.

Embodiment 36. The anti-CD147 antibody of any one of embodiments 33-35, wherein the Plasmodium parasite is selected from the group consisting of: Plasmodium falciparum, Plasmodium malariae, Plasmodium ovale, Plasmodium vivax, and Plasmodium knowlesi.

Embodiment 37. The anti-CD147 antibody of any one of embodiments 33-36, wherein the anti-CD147 antibody is effective in decreasing parasitemia in blood cells.

Embodiment 38. The anti-CD147 antibody of embodiment 37, wherein the anti-CD147 antibody decreases parasitemia in blood cells within about 48 hours.

Embodiment 39. The anti-CD147 antibody of embodiment 37, wherein the anti-CD147 antibody decreases parasitemia in blood cells within about 96 hours.

Embodiment 40. The anti-CD147 antibody of embodiment 33, wherein administration of the anti-CD147 antibody prevents, ameliorates, or treats malaria in a subject.

Embodiment 41. An anti-CD147 antibody that comprises a CDR1, a CDR2, and a CDR3 region, wherein the CDR1 region is an amino acid sequence selected from the group consisting of: SEQ ID NO: 16-SEQ ID NO: 23, wherein the CDR2 region is an amino acid sequence selected from the group consisting of: SEQ ID NO: 24-SEQ ID NO: 31, wherein the CDR3 region is an amino acid sequence selected from the group consisting of: SEQ ID NO: 32-SEQ ID NO: 39, and wherein the anti-CD147 antibody comprises from 0 to 5 amino acid modifications in at least one of the CDR1, CDR, or CDR3 regions.

Embodiment 42. The anti-CD147 antibody of embodiment 41, wherein the anti-CD147 antibody comprises from 0-3 amino acid modifications.

Embodiment 43. The anti-CD147 antibody of any one of embodiments 41-42, wherein the CDR1 region corresponds to SEQ ID NO: 18 or 22, wherein the CDR2 region corresponds to SEQ ID NO: 26 or 30, and wherein the CDR3 region corresponds to SEQ ID NO: 34 or 38.

Embodiment 44. The anti-CD147 antibody of any one of embodiments 41-43, wherein the anti-CD147 antibody comprises SEQ ID NO: 12 or SEQ ID NO: 14.

Embodiment 45. The anti-CD147 antibody of anyone of embodiments 41-44, wherein the anti-CD147 antibody is humanized.

Embodiment 46. A method of treatment, comprising administering an effective amount of a pharmaceutical composition comprising an anti-CD147 antibody, wherein the anti-CD147 antibody comprises a CDR1, a CDR2, and a CDR3 region, wherein the CDR1 region is an amino acid sequence selected from the group consisting of: SEQ ID NO: 16-SEQ ID NO: 23, wherein the CDR2 region is an amino acid sequence selected from the group consisting of: SEQ ID NO: 24-SEQ ID NO: 31, wherein the CDR3 region is an amino acid sequence selected from the group consisting of: SEQ ID NO: 32-SEQ ID NO: 39, and wherein the anti-CD147 antibody comprises from 0 to 2 amino acid modifications in at least one of the CDR1, CDR, or CDR3 regions.

Embodiment 47. The method of treatment of embodiment 46, wherein the administering is effective in reducing or eliminating an inflammatory or autoimmune disease.

Embodiment 48. The method of treatment of any one of embodiments 46-47, wherein the inflammatory or autoimmune disease is selected from the group consisting of: rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

Embodiment 49. The method of treatment of any one of embodiments 46-48, wherein the administering is effective in reducing or eliminating viral invasion of a cell by a virus.

Embodiment 50. The method of treatment of embodiment 49, wherein the virus is selected from the group consisting of measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis.

Embodiment 51. The method of treatment of embodiment 50, wherein the virus is the coronavirus.

Embodiment 52. The method of treatment of embodiment 51, wherein the coronavirus comprises SARS-CoV-2.

Embodiment 53. The method of treatment of any one of embodiments 46-48, wherein the administering is effective in reducing or eliminating metastasis of a cancer.

Embodiment 54. The method of treatment of embodiment 53, wherein the cancer is selected from the group consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

Embodiment 55. The method of treatment of any one of embodiments 46-48, wherein the administering is effective in reducing or eliminating invasion of Plasmodium into a cell.

Embodiment 56. An antibody-drug conjugate that comprises the anti-CD147 antibody of any one of embodiments 1-29.

Embodiment 57. The antibody-drug conjugate of embodiment 56, wherein the drug is selected from the group consisting of an: anti-cancer drug, anti-malaria drug, anti-viral drug, and an anti-inflammatory drug.

Embodiment 58. The antibody-drug conjugate of embodiment 57, comprising the anti-viral drug, wherein the anti-viral drug is an anti-SARS-CoV-2 drug.

Embodiment 59. The antibody-drug conjugate of embodiment 57, comprising the anti-cancer drug, wherein the anti-cancer drug comprises an immunotherapy.

Embodiment 60. The antibody-drug conjugate of embodiment 59, wherein the immunotherapy is selected from the group consisting of an antibody, checkpoint inhibitor, cell therapy, cytokine, oncolytic virus, and vaccine.

Embodiment 61. The antibody-drug conjugate of embodiment 60, comprising the antibody.

Embodiment 62. The antibody-drug conjugate of embodiment 56, wherein the anti-CD147 antibody comprises SEQ ID NO: 12 or SEQ ID NO: 14.

### EXAMPLES

### Example 1—Production of VhH Antibodies that Bind Human CD147

To create VhH antibodies that bind human CD147, a llama was immunized with recombinant CD147 (in a cocktail of 21 antigens) and boosted four times. mRNA was extracted from blood lymphocytes, and a VhH antibody display library was constructed in a yeast surface expression system. Yeast clones expressing anti-CD147 VhH antibodies were enriched by two rounds of panning and FACS, and twenty-four were analyzed for VhH secretion. Their anti-CD147 activity was measured by ELISA.

Of these 24, four clones with unique sequences and high signal to noise ratios on ELISA were selected for further study. Two of these (Ibx-11 and Ibx-13) expressed well in mammalian cells as IgG Fc fusions, and three tandem bivalent VhH constructions (without IgG Fc fusions) were also produced (Ibx-75, Ibx-76, Ibx-75 VhH).

For the ELISA, 100 ng of human CD147 was coated onto Nunc polysorb plates overnight in 1× coating buffer. The wells were washed once with TBST (0.01% tween 20) and then blocked for 1 hour using 1× fishgel in TBST at room temperature (RT). Ibx-11, Ibx-13, Ibx-75, Ibx-76, Ibx-77, and anti-HIS antibody was incubated at 0.01-100 ng/well in 100 μl for 1 hour at RT before being washed 3× for 5 min each wash. The wells were incubated with anti-human for Ibx-11 & Ibx-13 treated wells and with anti-flag for Ibx-77, Ibx-75, Ibx-76 treated antibodies at 1:1000 dilution for 1 hour RT, and then washed 3× for 5 min each wash. The wells were incubated with 100 μl/Well of TMBE for 30 minutes and the reaction was stopped with 100 μl/Well of 2N sulphuric acid. Optical Density (OD) was measured using Biotek synergy 4 instrument for quantification.

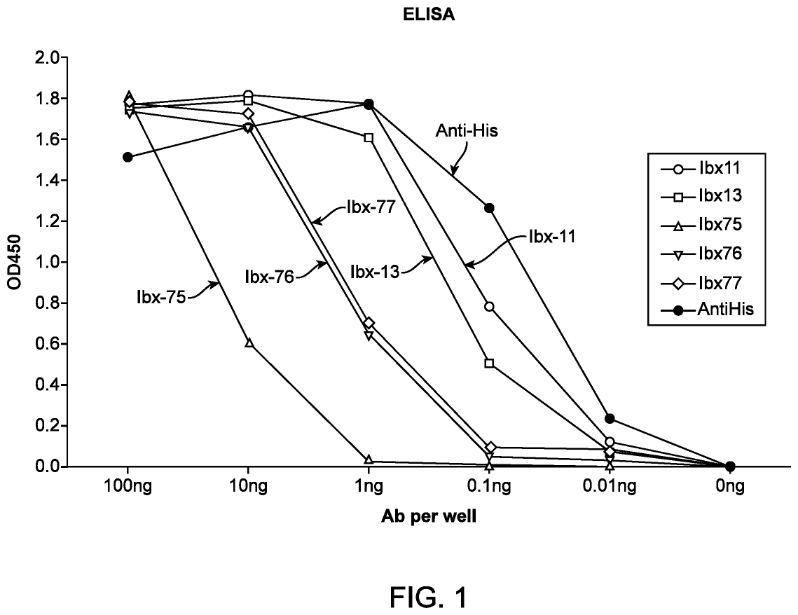

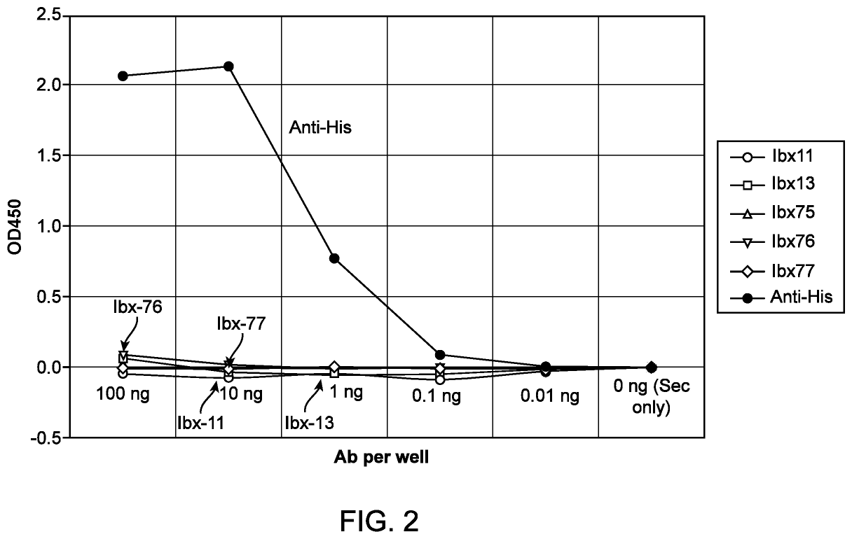

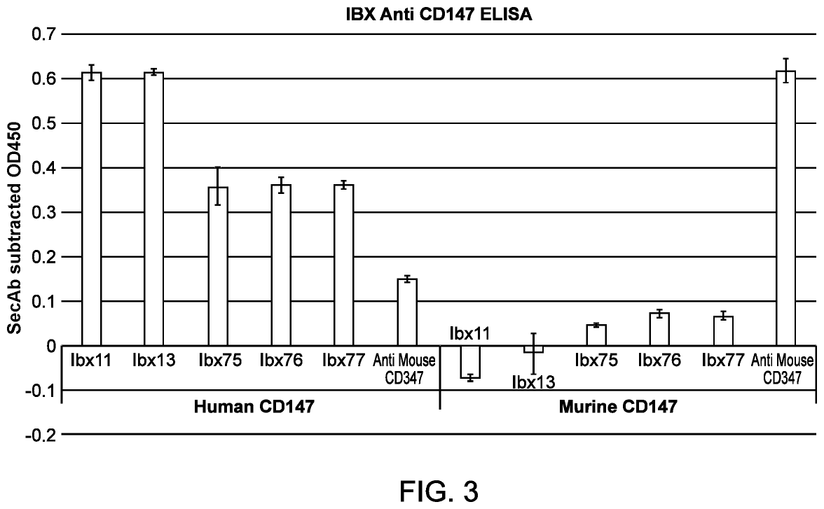

The results from this assay are shown in FIG. 1. The ELISA EC50 for the tested antibodies ranged over two logs (0.1-10 ng VhH antibody/well). The tandem bivalent Ibx-75 (10 ng/well) showed the lowest binding to human CD147 followed by Ibx-76 and Ibx-77 which bound at the same level (1 ng/well). The best binding was observed with the IgG Fc fusions Ibx-11 and Ibx-13 (0.1 ng/well). The improved binding of the IgG Fc fusions over the tandem bivalents may be due to different secondary reagents used (anti-Fc vs anti-Flag, respectively), or differences in functional valency (i.e., the tandem bivalent constructions may be effectively binding as a monovalent structure due to short linker length).

### Example 2—Binding of VhHs to Murine CD147

To test the ability of the VhHs described in Example 1 to bind to murine CD147, an ELISA was performed.

For the ELISA, 100 ng of murine CD147 was coated onto Nunc polysorb plates overnight in 1× coating buffer. The wells were washed once with TBST (0.01% tween 20) and then blocked for 1 hour using 1× fishgel in TBST at room temperature (RT). Ibx-11, Ibx-13, Ibx-75, Ibx-76, Ibx-77, and anti-HIS antibody was incubated at 0.01-100 ng/well in 100 μl for 1 hour at RT before being washed 3× for 5 min each wash. The wells were incubated with anti-human for Ibx-11 & Ibx-13 treated wells and with anti-flag for Ibx-77, Ibx-75, Ibx-76 treated wells at 1:1000 dilution for 1 hour RT, and then washed 3× for 5 min each wash. The wells were incubated with 100p/Well of TMBE for 30 minutes and the reaction was stopped with 100p/Well of 2N sulphuric acid. Optical Density (OD) was measured using Biotek synergy 4 instrument for quantification.

### Example 3—SPR Analysis of IBX-77 Binding to CD147

For this assay, CD147 was injected in HBS-EP buffer pH7.4 at concentrations ranging from 0 nM to 900 nM at 10 μl/min for 600 Seconds. Duplicate CD147 injections were carried out for 0, 9.22, 0, 23.04, 57.6, 144, 360, 900 nM (a 2.5-fold dilution series). The chip surface was regenerated using 10 mM Glycine pH 2.0 @ 10 μl/min for 180 sec.

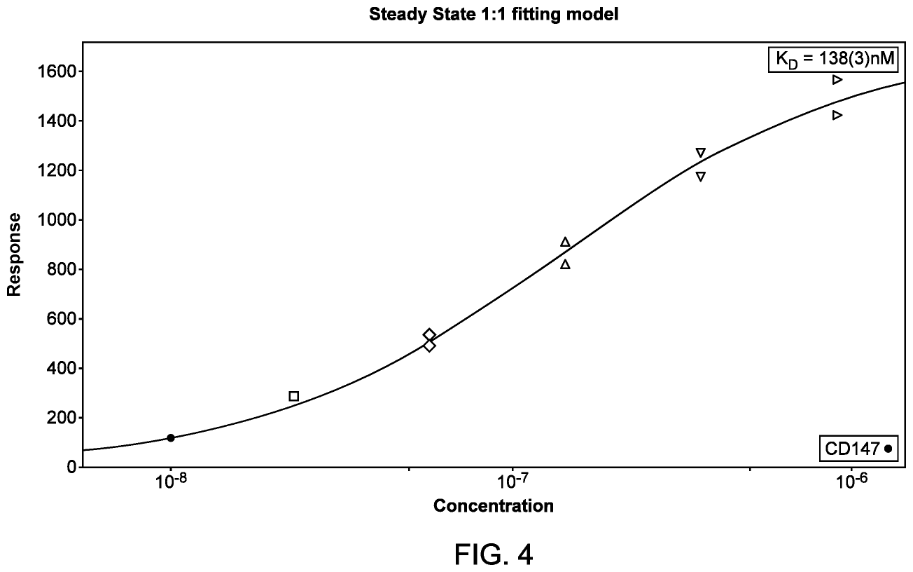

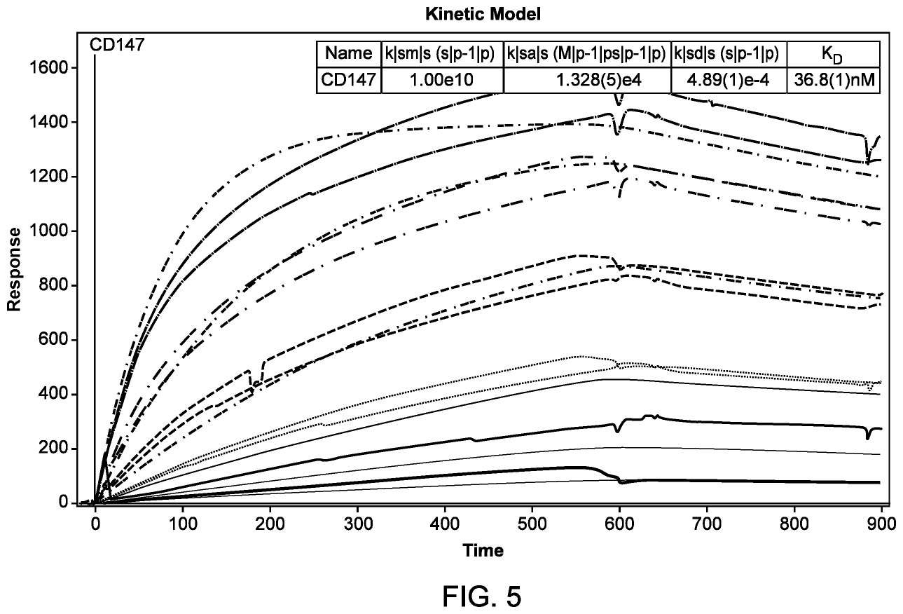

One technical replicate of 0 nm & 23.04 nM was excluded from analysis due to sudden air spikes and DIPs during analyte injection. Data were analyzed in Q-Dat software provided by instrument manufacturer. A steady state 1:1 fitting model (FIG. 4) was used for initial steady state binding analysis, and a simple Kd/Ka kinetic model (FIG. 5) was used to estimate KD.

The duplicate injections for each concentration were similar but the response the second time was lower than first. The Steady state 1:1 calculated KD=138.0 nM (±0.003 nM). The Kinetic model calculated

Ka=1.328×10⁴ (±0.005) M-1s-1

Kd=4.89×10⁻⁴ (±0.001) s-1

KD=36.1 nM (±0.001 nM).

### Example 4—SPR Analysis of Ibx-77 Binding to CD147 on CMD Chip

The CMD chip surface was activated by injecting 1:1 mixture of NHS/EDC for 420 sec at 10 μl/min. 100 μg/ml CD147 was immobilized with 10 mM Sodium Acetate solution pH4.5 at 10 μl/min for 510 sec. The unreacted CD147 and activated chip surface were washed and inactivated by ethanolamine pH8.5 at 10 μl/min for 420 sec. A total of approx. 3800 RU of CD147 was immobilized.

Ibx-77 was injected in HBS-EP buffer pH 7.4 at concentrations ranging from 9.22 nM to 360 nM at 10 μl/min for 420 sec. The chip surface was regenerated using 10 mM Glycine pH 2.0

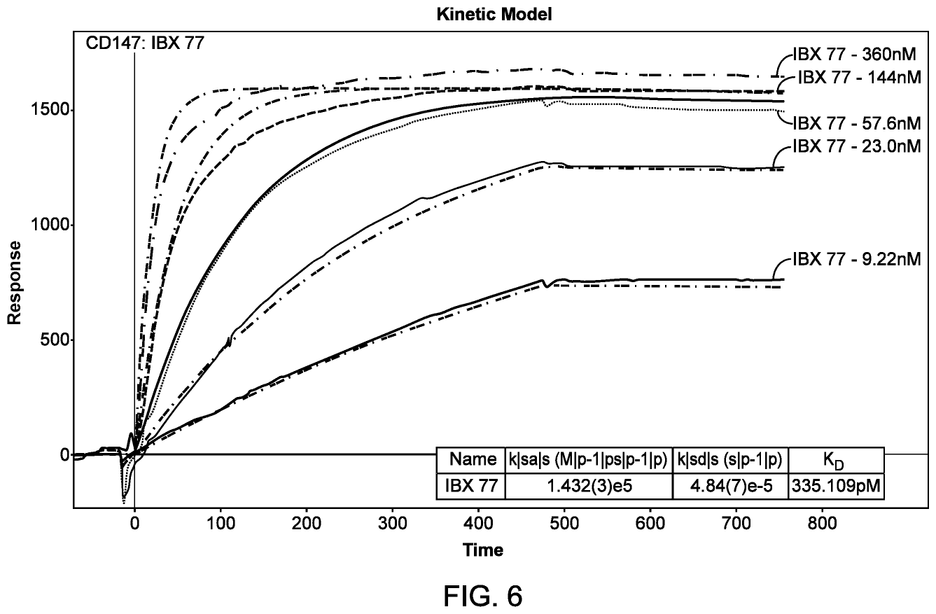

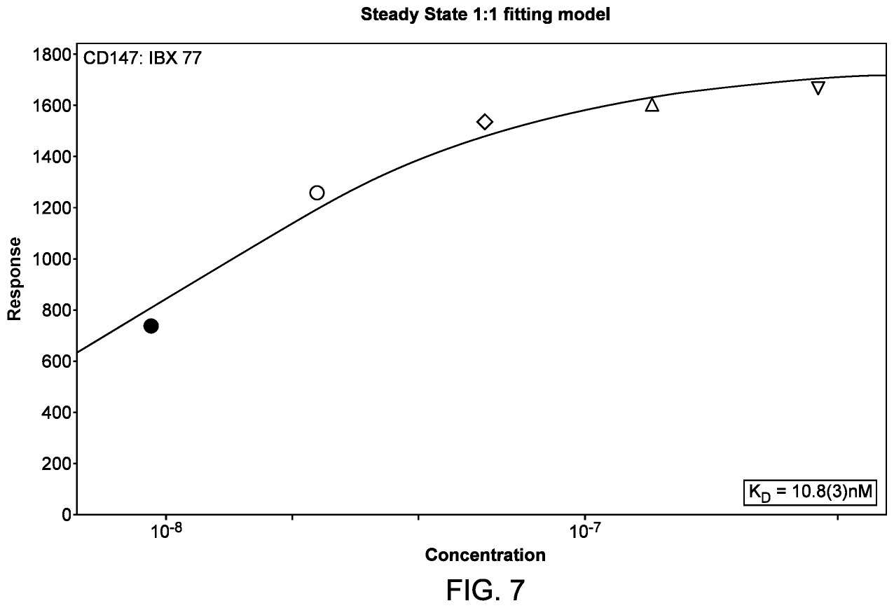

Data was analyzed in qDat software provided by instrument manufacturer. A steady state 1:1 fitting model was used for initial steady state binding analysis. (FIG. 7). A simple Kd/Ka kinetic model was then used to estimate KD. (FIG. 6).

### Example 5—Ibx-77 Binds to HEK 293 and HCC 1954 Cells

To test the ability of the VhH antibodies of the disclosure to bind to CD147 expressed on cells, flow cytometry was performed. All cell lines were grown to confluency in recommended medium in T-75 flask. (HCC1954—RPMI; SKBR3—McCoy's 5A; HEK 293—DMEM; MCF-7—EMEM). Approximately 1 million cells were plated on to 6 well plates and allowed to settle and grow overnight. Cells were lifted by manual scrapping with wide blunt mouth end of 1 ml pipette tip and Cells were prepared for flow cytometry analysis as before. 1 μg of Ibx-77, anti-CD147, Rabbit isotype control, anti-Flag dylight were used for staining cells, for 1 hour on ice, followed by wash and centrifugation. Then the cells were incubated for 30 min with 1 μg anti-rabbit Dylight488 as secondary and anti-Flag dyligth488, followed by centrifugation and wash. The cells were resuspended in 1000 μl of flow sauce for final run-on flow cytometer. 30,000 total events were collected for analysis wherever possible within 300 sec (5 min). Negative cells were gated out based on the unstained cells for individual cell line.

Conditions for each cell line: CST for positive control only; Ibx-77 only; ISO type controls rabbit; UST; Anti-Flag 488 2° only—XF2.

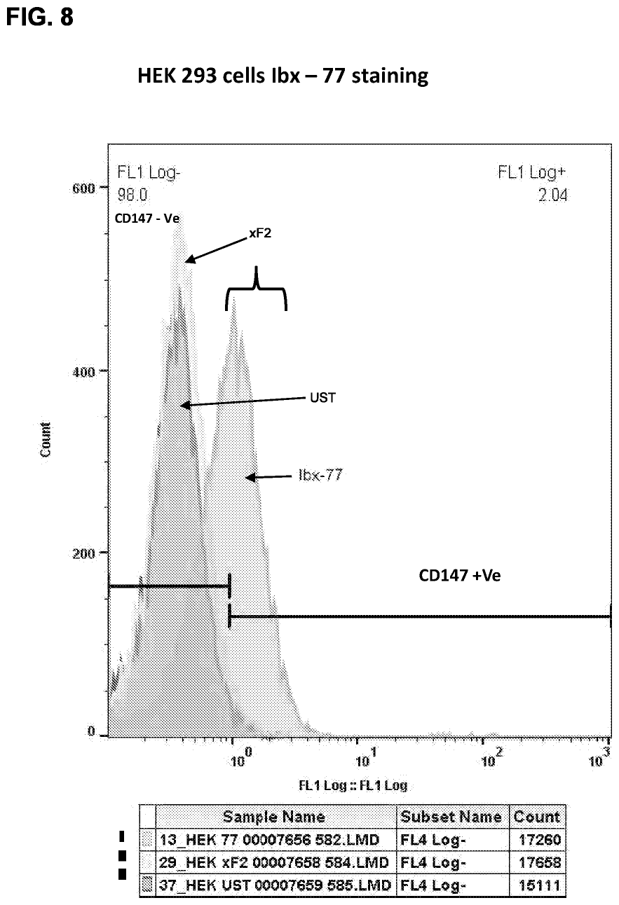

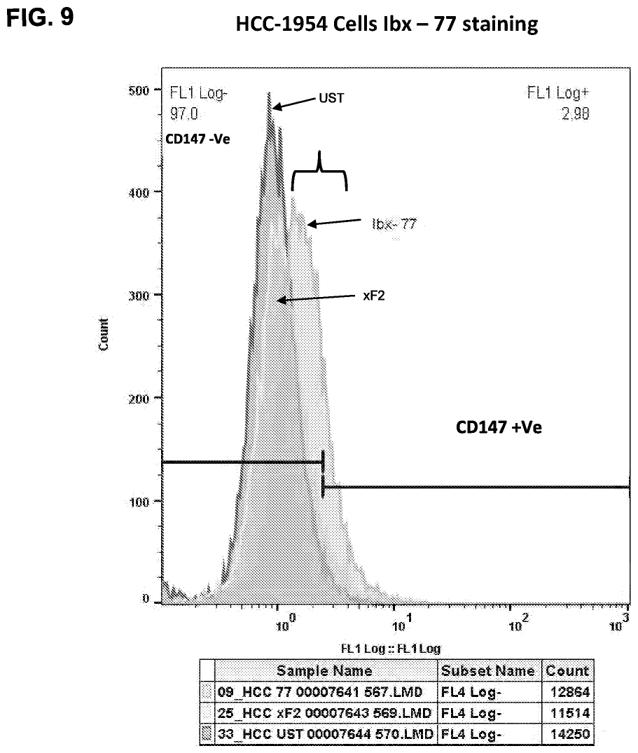

Ibx-77-stained cells show positive stained cell populations that are distinct from unstained controls in the HEK 293 cells (FIG. 8) and HCC 1954 (FIG. 9) cell lines.

### Example 6—Ibx-77 Binds to CD147 Expressed on HeLa Cells

HELA cell lines were grown to confluency in recommended medium in T-75 flask. Approximately 1 million cells were plated on to 6 well plates and were allowed to settle and grow overnight. Cells were lifted by manual scrapping with wide blunt mouth end of 1 ml pipette tip and Cells were prepared for flow cytometry analysis as before. Cells were washed once in flow sauce, incubated in 1 μl/ml viability dye 660 for 30 minutes on ice, then washed 2 times. 10 μg and 5 μg of Ibx-77 were used for staining cells, for 1 hour on ice, followed by 2× washes. Then the cells were incubated for 30 min with 1p g anti human Dylight488 as secondary and anti-Flag dyligth488, followed 2× washes. The cells were resuspended in 1000 μl of flow sauce for final run-on flow cytometer. 50,000 total events were collected for analysis wherever possible within 300 seconds (5 minutes). Negative cells were gated out based on the unstained cells for the individual cell line. Conditions for each cell line were: Ibx-77 only; UST, Anti flag 488 secondary only—Flag 2 only.

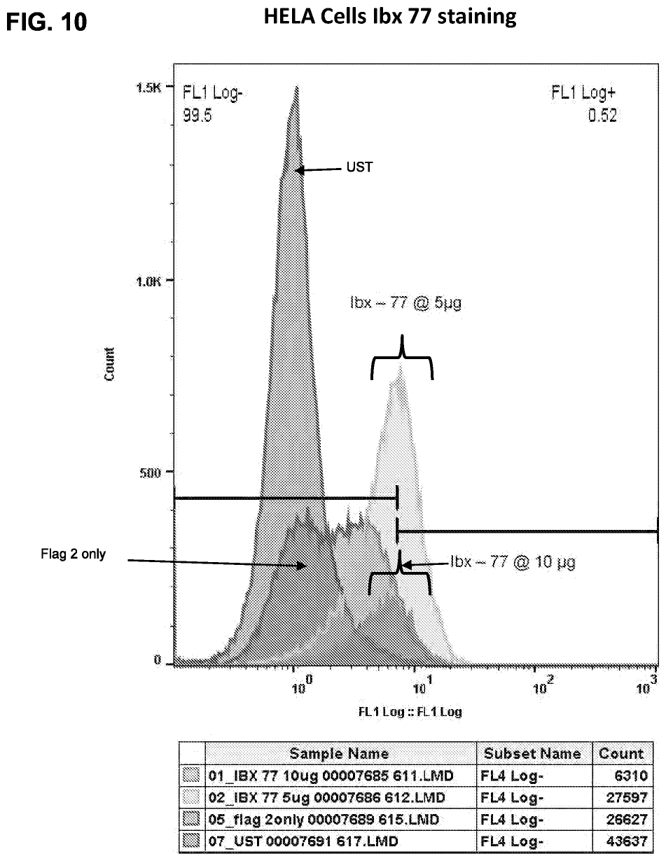

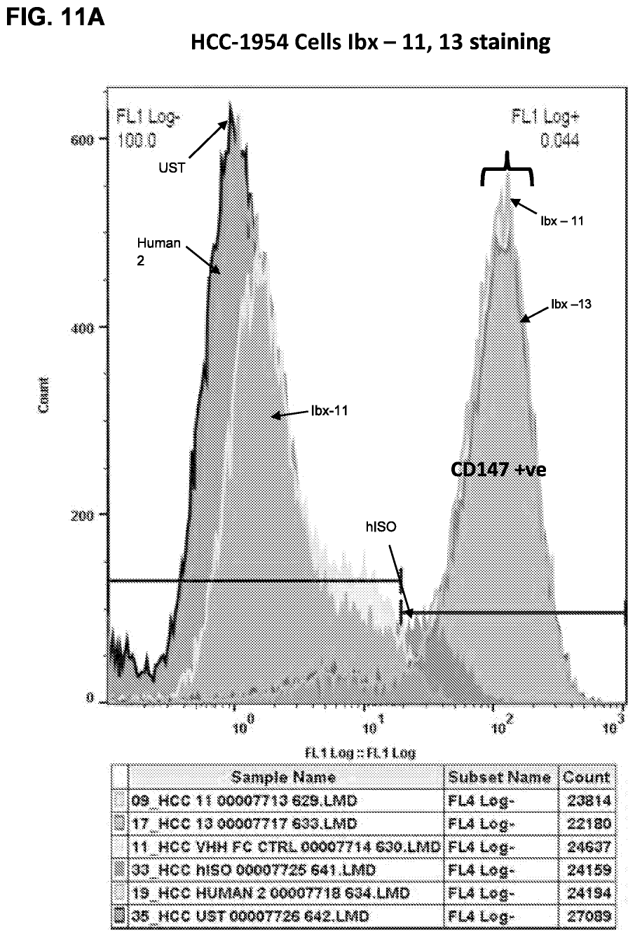

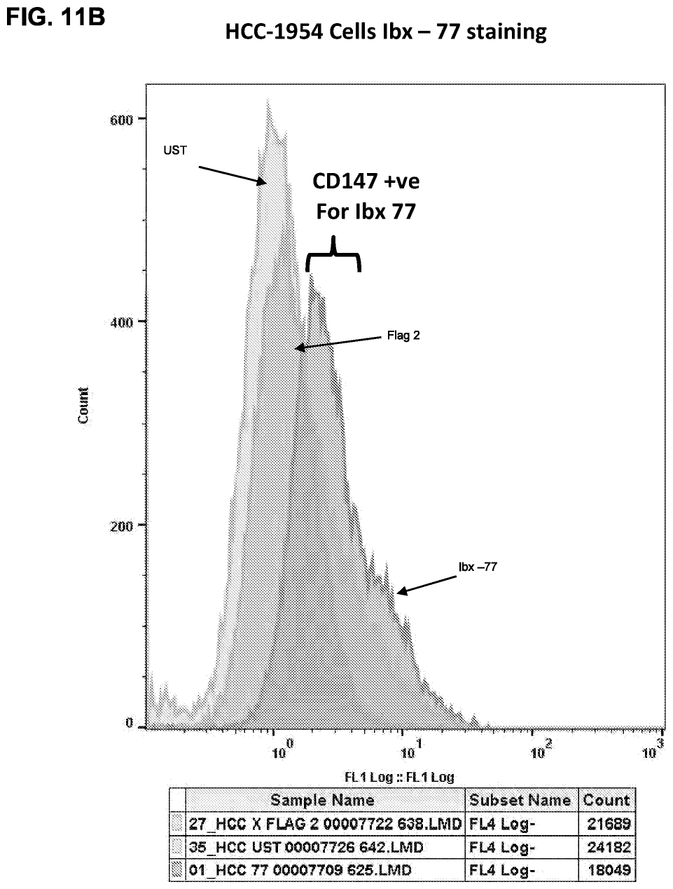

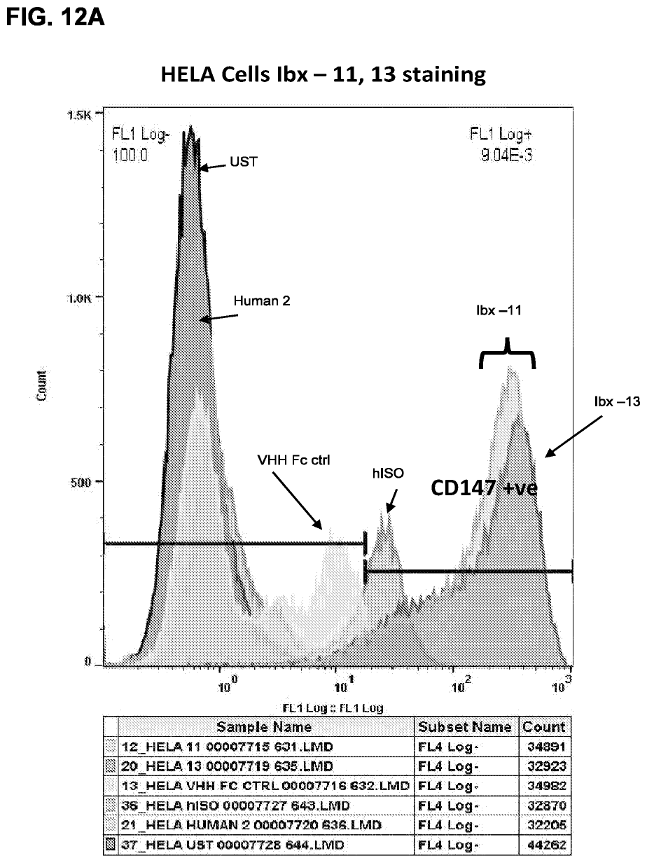

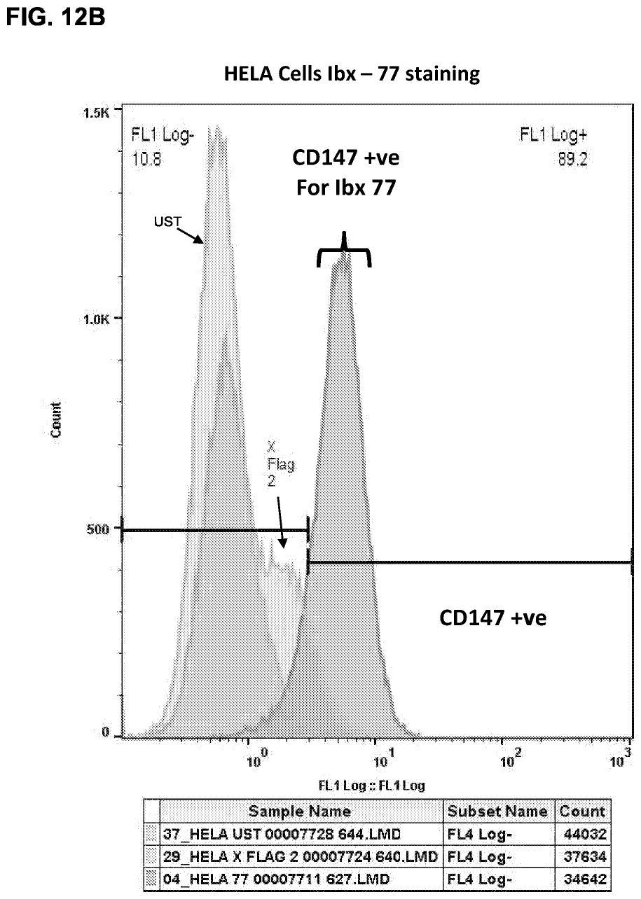

As shown in FIG. 10, 5 μg of Ibx-77 shows a peak that is different from secondary only control and unstained cells.

### Example 7-Analysis of the Ability of Ibx-77, Ibx-13, and Ibx-11 to Bind to Cells

HeLa and HCC-1954 cell lines were grown to confluency in recommended medium in T-75 flask. Approximately 1 million cells were plated on to 6 well plates and were allowed to settle and grow overnight. Cells were lifted by manual scrapping with wide blunt mouth end of 1 ml pipette tip and Cells were prepared for flow cytometry analysis as before. Cells were washed once in flow sauce (PBS, 1% FBS and Na Azide). Incubated in 1 μl/ml viability dye 660 for 30 min on ice, then washed twice. 1p g/100 μl of Fc block reagent was used to block the probable Fc receptors for 10 min on ice. Followed by washing twice. Antibodies in indicated quantitates as shown in table were used for staining cells, for 1 hr on ice, followed by 2 washes. Then the cells were incubated for 30 minutes with anti-human Dylight488 as secondary, anti-rabbit Dylight488 and anti-Flag Dyligth488, followed twice with washes. The cells were resuspended in 2000 μl of flow sauce for a final run on the flow cytometer. 50,000 total events were collected for analysis wherever possible within 300 seconds (5 minutes). Negative cells were gated out based on the VhH-Fc-(h27-8GL) cells for individual cell lines.

The following antibodies and controls were tested in this experiment: Ibx-77 (10 μg); Ibx-13 (10 μg); Ibx-11 (10 μg); human isotype control (10 μg); anti-human secondary only (1 μg; Human 2); VhH-Fc-(h27-8GL) (10 μg); XF2 (anti-flag 488) only; unstained.

Ibx-11 and Ibx-13 stain cells in a similar pattern and the histograms are imposable. Ibx-77 shows weaker staining probably due to one Flag epitope available for the secondary antibody to bind to. Blocking with 1 μg of Fc block reduced the background noise of the human isotype controls a bit. VhH-Fc (h27-8GL) control antibody clearly shows the difference in staining between itself and Ibx-11 and Ibx-13. So, for every experiment that uses VhH-Fc, such a control could be used. Overall HELA cells appear to express more CD147 than HCC 1954.

### Example 8—Analysis of the Ability of Ibx-11 and Ibex-13 Antibodies to Bind to Jurkat T-Cells

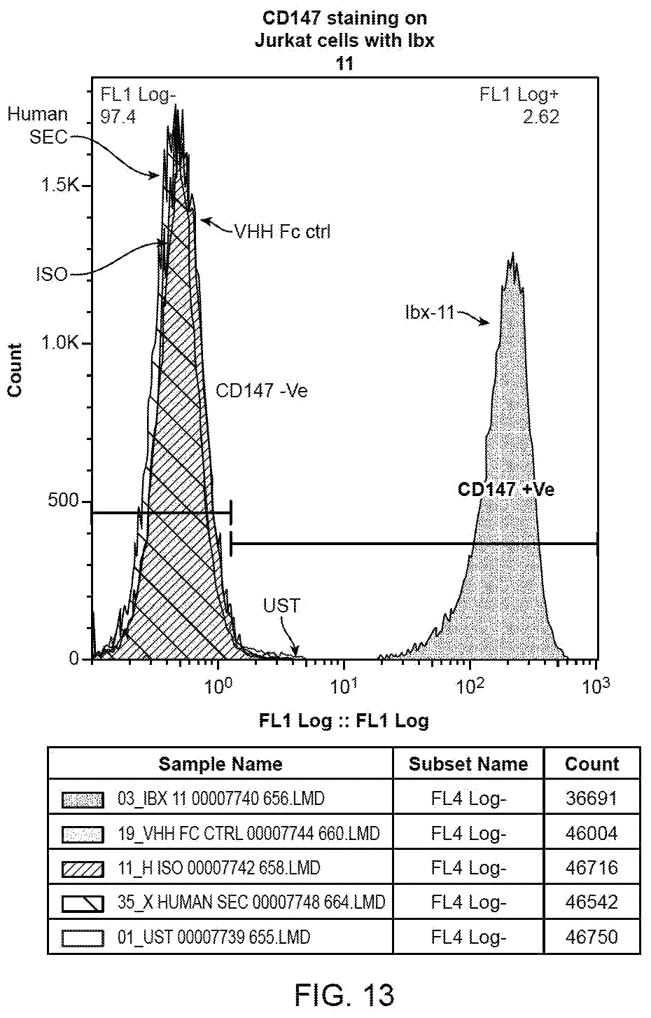

Jurkat cells were grown to confluency in recommended medium, and approximately 4 million cells were collected and prepared for flow cytometry analysis as before. These cells were not Na Azide-treated. The cells were washed once in flow sauce (PBS, 1% FBS and Na Azide) before incubation in 1 μl/mL viability dye 660 for 30 minutes on ice and washed twice. One μg/100 μl of Fc bloc reagent was used to block Fc receptors for 10 minutes at room temperature, followed by two washes. The cells were stained with antibodies in the indicated quantities as shown in the table in FIG. 13 for one hour on ice, followed by two washes. The designated tubes were incubated with spike RBD (1 μg/tube) for one hour and then washed twice. The cells were then incubated with anti-spike antibody, VhH-Fc control, human isotype antibody or Ibx-11 for one hour at 4° C., and then washed twice. The cells were then incubated for 30 minutes with anti-human Dylight488 and then anti-rabbit Dylight488 followed by two washes. The cells were resuspended in 1000 μl of flow sauce for a final run on the flow cytometer. 50,000 total events were collected for analysis wherever possible within 300 seconds (5 minutes). Negative cells were gated out based on the VhH-Fc(h27-8GL) cells for individual cell lines.

Overall, the Ibx-11 and Ibex-13 antibodies stained the Jurkat cells.

### Example 9—Analysis of the Ability of Ibx-11 and Ibex-13 Antibodies to Bind to Vero E-6 Cells

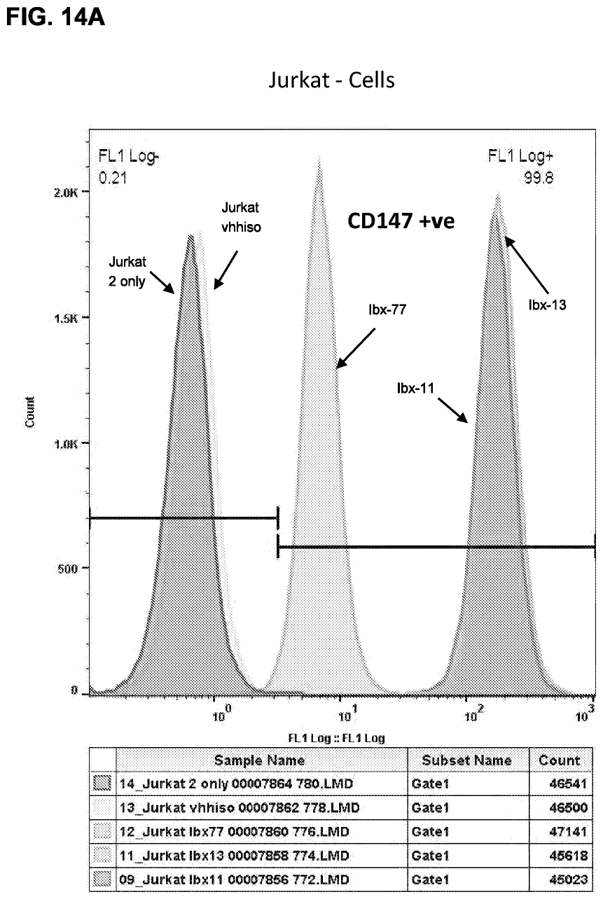

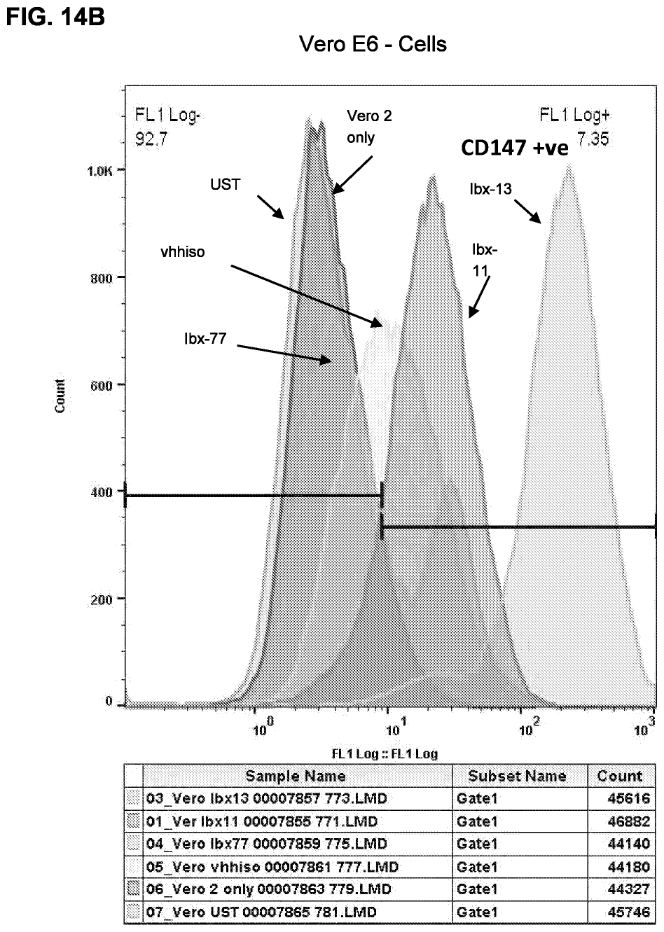

Jurkat and Vero E-6 cells were grown to confluency in recommended medium. Approximately 1 million cells per well were plated on a 6 well-dish and were prepare for flow cytometry analysis as before. The cells were not Na-Azide treated. The cells were washed once in flow sauce (PBS, 1% FBS, and Na Azide). One μg/100 μl of Fc bloc reagent was used to block the Fc receptors for 10 minutes at room temperature, followed by two washes. The cells were stained with antibodies in the indicated quantities as shown in the table in FIG. 14A-B for one hour on ice, followed by two washes. Cells were then incubated with spike RBD (1 μg/tube) for one hour and then washed twice. The cells were then incubated with anti-spike antibody, VhH-Fc control, human isotype antibody or Ibx-11 Ibx-13, or Ibx-77 for one hour at 4° C., and then washed twice. The cells were then incubated for 30 minutes with anti-human Dylight488 and then anti-rabbit Dylight488 followed by two washes. The cells were resuspended in 1000 μl of flow sauce for a final run on the flow cytometer. 50,000 total events were collected for analysis wherever possible within 300 seconds (5 minutes). Negative cells were gated out based on the VhH-Fc(h27-8GL) cells for individual cell lines.

The Vero E6 cells were analyzed using the same voltage settings on the flow cytometer that were set for Jurkat Cells. Jurkat cells show good staining with Ibx-11, Ibx-13 and Ibx-77 with distinct separation between the negative (VhH-Fc-(h27-8GL) and unstained controls. See FIG. 14A. Surprisingly, the Vero E6 cells show that Ibx-13 stains stronger than Ibx-11. See FIG. 14B. These differences could be because of differences in sequence of human and monkey CD147. Such differences in amino acids may be part of the critical epitopes for the Ibx antibody series and can used for potential epitope mapping. For nonhuman primate models, Ibx-13 and its dimerized version Ibx-76 may be good candidates. Ibx-77 showed two peak populations that may indicate weaker staining on the Vero E6 cells.

### Example 10—KD of Ibx-74 and Ibx-75

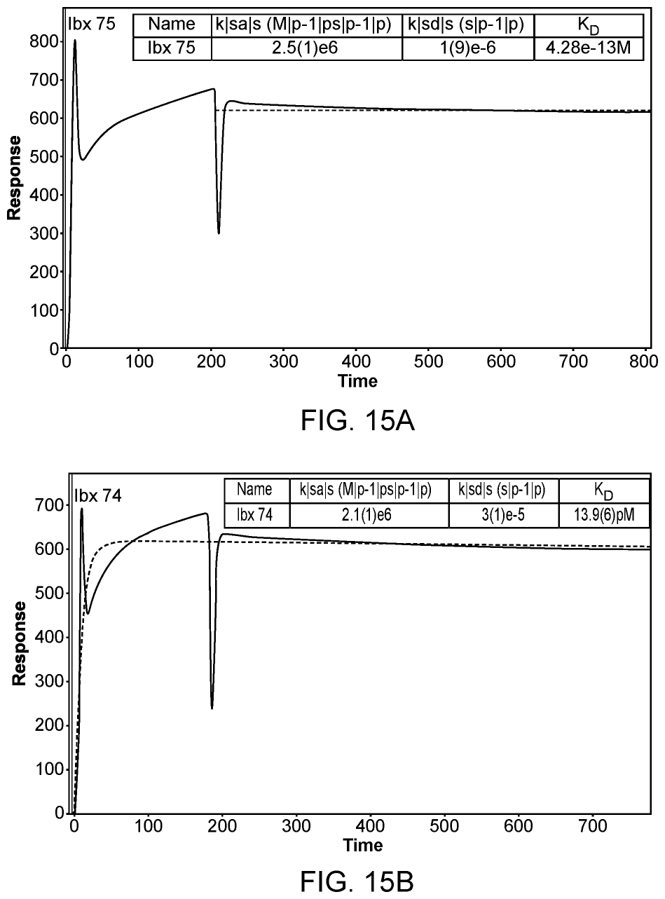

Approximately 45 μl of Ibx-74 and Ibx-75 at various stages of purification were provided; 35 μl of each sample was injected on a sensor chip and sensograms were collected. The flow rate was 10 μ/minute, and the chip was regenerated with 10 mM Glycine at pH 1.5. The results are shown in FIGS. 15A-B.

While the KD values obtained here may be influenced by impurities or concentration, the values depend mostly on the antibody, and can therefore be used as a screening tool to identify strong binders. Here, Ibx-75 bound with a K_{d} of 1×10⁻⁶ (+0.009) s⁻¹ and Ibx-74 bound with a K_{d} of 3×10⁻⁵ (±0.001) s⁻¹

This comparison shows that Ibx-75 may be a stronger binder with lower off rates than Ibx-74.

### Example 11—Antibody-Drug Conjugate (ADC) Cytotoxic Assay

MiaPaCa-2 cancer cells were seeded 2500 cells/well in a 96 well dish, allowed to adhere, and grown overnight. Old medium was replaced with 50 μl of fresh medium. Ibx-11 and Ibx-13 were prepared by premixing the antibody with anti-human IgG Fc-MMAF. Fifty microliters of the Ibx-11 and Ibx-13 mixtures in various concentrations (final concentrations of 30 nM to 1.1 nM) was added to the wells. Isotype and secondary ADC only controls were included on the plates. The plates were then incubated for 72 hours. After this 72-hour incubation, the viability of the cells was measured using Rockland Kit.

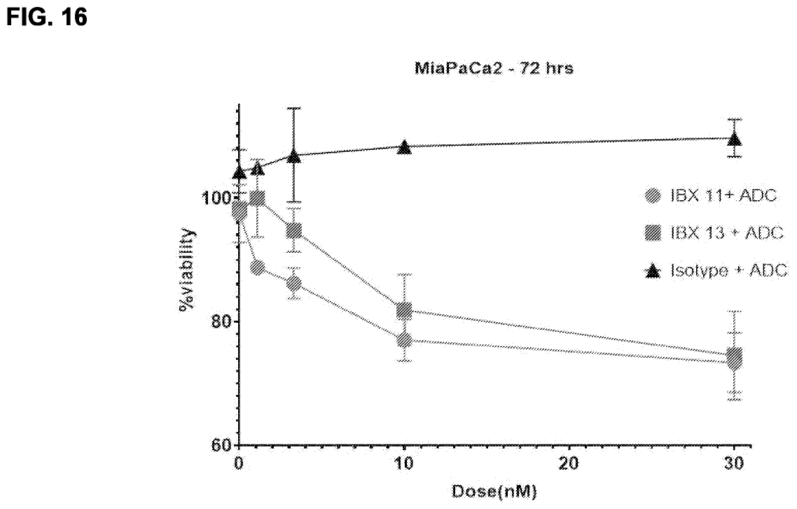

At the highest concentration, Ibx-11 and Ibx-13 showed about 73% and 74% viable cells respectively. See FIG. 16. These antibodies effectively killed the cancer cells.

### Example 12—Jurkat T-Cell Aggregation Assay

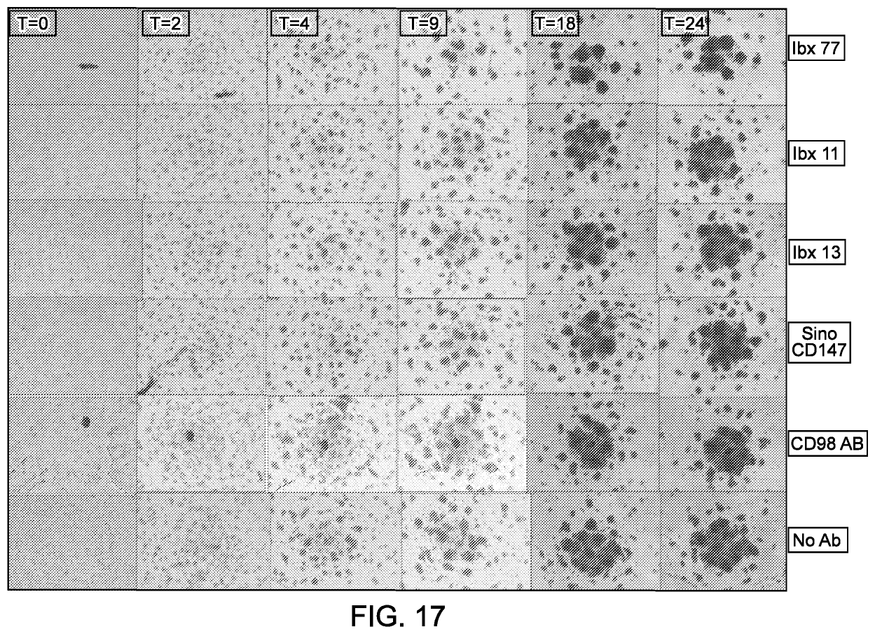

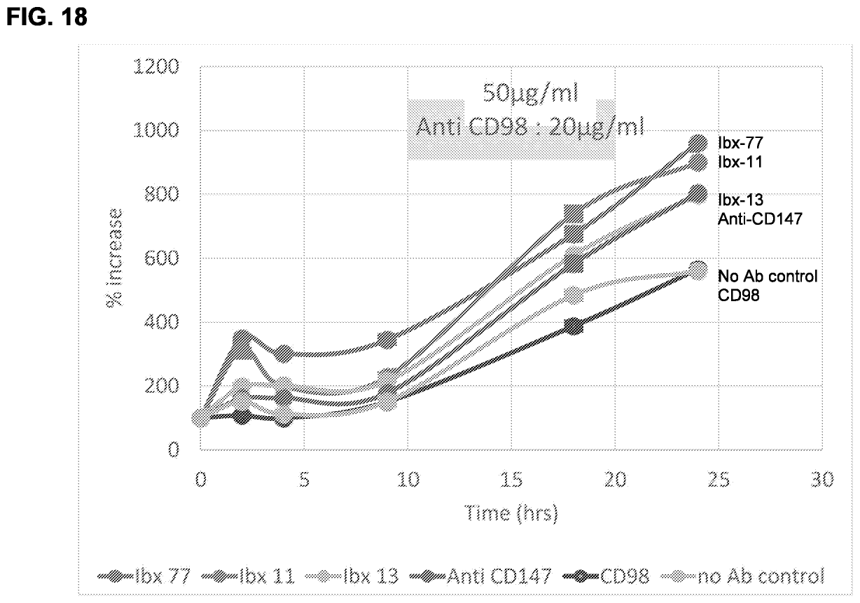

Jurkat T-cells were plated at 20,000 cells/well in a 96 round bottom well in 100 μl. 100 μg/ml of Ibx-77, Ibx-11, or Ibx-13 were added to the cells in 100 μl to a final volume of 200 μl per well and a final concentration of 50 μg/ml antibodies. Images were collected at 0 hours, 2 hours, 4 hours, 9 hours, 18 hours, and 24 hours. ImageJ was used for measuring pixel 2 hours and reached a maximum at 24 hours. No further differences were noticed at 48 hours. Visual identification (FIG. 17) shows differences in the pattern of aggregation was evident at 18 hours, with cells treated with an Ibx series antibody at 50 μg/ml showing distinct aggregation patterns with tighter clusters of aggregates and fewer individual cells, compared to untreated control A similar pattern was observed with the anti-CD147 control antibody and the anti-CD98 antibody at lower doses of 20 μg/ml. These data were also analyzed quantitatively with imaging analysis which counts the pixel size from an 8-bit image. 50 μg/ml-25 μg/ml Ibx-series antibodies show larger pixel sizes for the particles (aggregates) at 24 hours. These differences are subtle between Ibx-series antibodies. See FIG. 18.

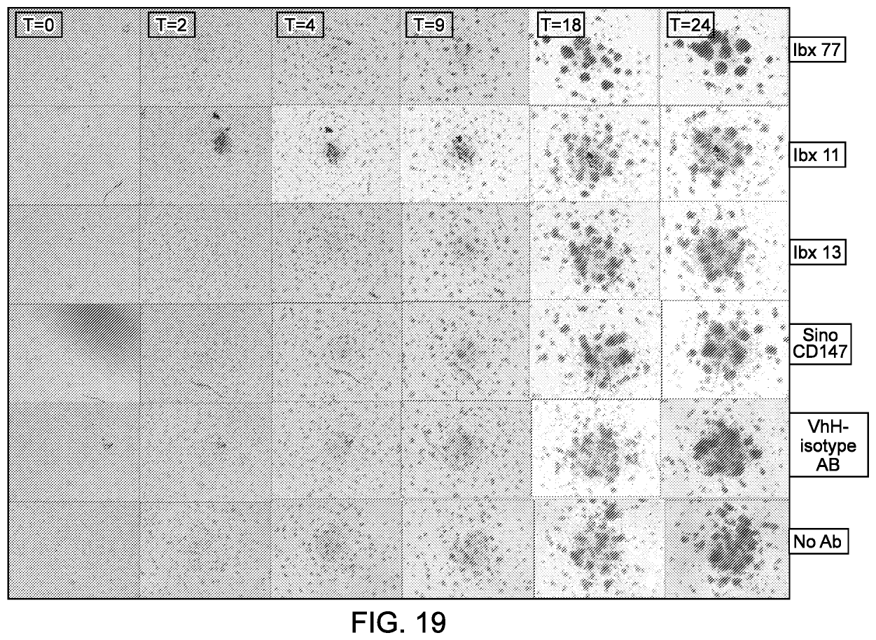

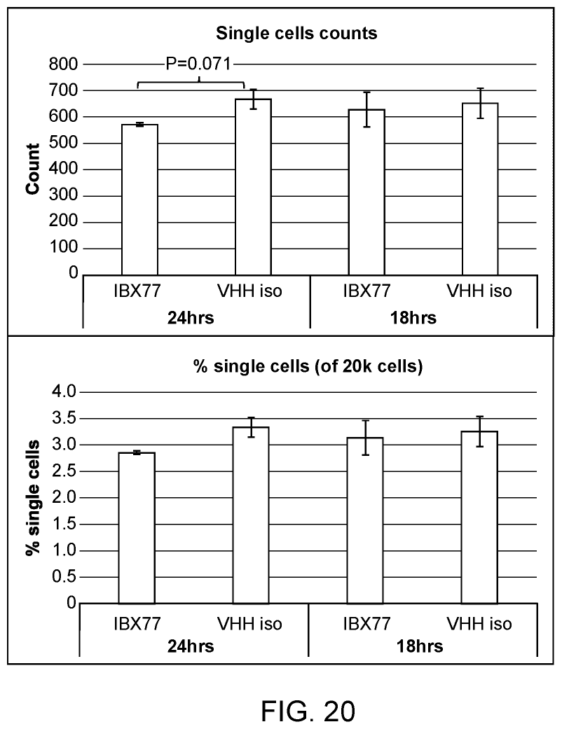

In another cell-aggregation assay, Jurkat T-cells were plated at 20,000 cells/well in a 96 round bottom well in 100 μl. 100 μg/ml of Ibx-77, Ibx-11, or Ibx-13 were added to the cells in 100 μl to a final volume of 200 μl per well and a final concentration of 50 μg/ml antibodies. Images were collected at 0 hours, 2 hours, 4 hours, 9 hours, 18 hours, and 24 hours. Individual cells and groups of cells (less than 3 cells per group) that were not part of the larger aggregates, with clear distinct borders were counted manually at 18 hours and 24 hours. FIG. 19. At 24 hours, there was a significant decrease in single cell counts for Ibx-77 treated cells (p=0.071). FIG. 20A-B.

### Example 13—Ibx-76 and Ibx-77 Inhibit Angiogenesis in Tubule Formation Assay

The ability of the anti-CD147 VhH antibodies to inhibit angiogenesis was explored using a tubule formation assay. HuVEC cells were seeded onto the surface of Matrigel coated wells. Antibodies and CD147 protein (added for competition) were added the day after seeding. After an additional day of culture, cells were stained with Calcein AM. Tube formation was then assessed by microscopy. A mouse antibody that binds human CD147 (HIM6) was used here as a positive control.

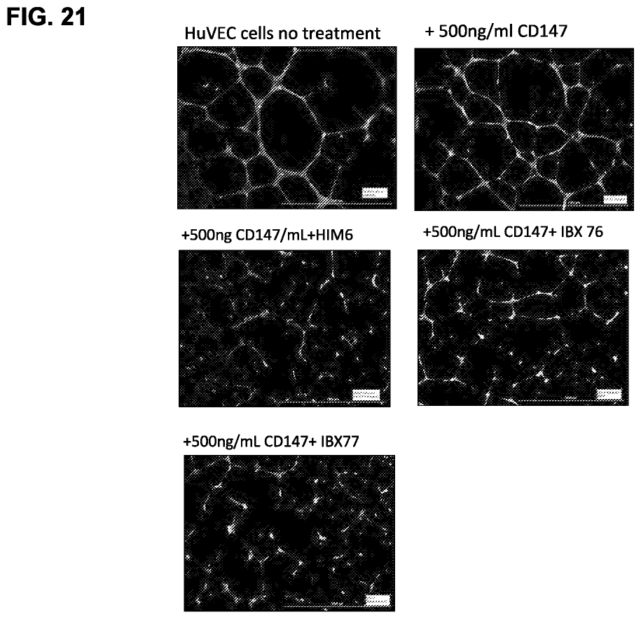

As shown in FIG. 21, cells treated with either Ibx-76 or Ibx-77 showed an inhibition of angiogenesis. Here CD147 is used as a stimulant for angio-genesis. Co-incubation of CD147 with Ibx-76 and Ibx-77 shows a decrease in the angiogenesis that CD147 alone would induce.

### Example 14—Ibx-13 Prevents Growth of Plasmodium falciparum in Blood Cells

The ability of different antibodies to block the growth of the NF54 strain of Plasmodium falciparum: in red blood cells. The initial parasitemia was 1.3% and the strain was cultured at 2% hematocrit in a 96-well plate. The Ibx-13 or MIF-2-5 antibodies or VhH-Fc were administered to the wells at 1, 10, and 100 μg/mL. Parasites not exposed to any antibody were cultured as the negative control, and the parasites cultured with the anti-malarial agent chloroquine (CQ) were used as the positive control. Blood smears were prepared at 48- and 96-hours post-treatment, stained, and ready by microscopy in a blinded manner. The number of infected red blood cells (RBCs) were scored in a blinded manner out of approximately 1000 red blood cells and expressed as a percentage parasitemia.

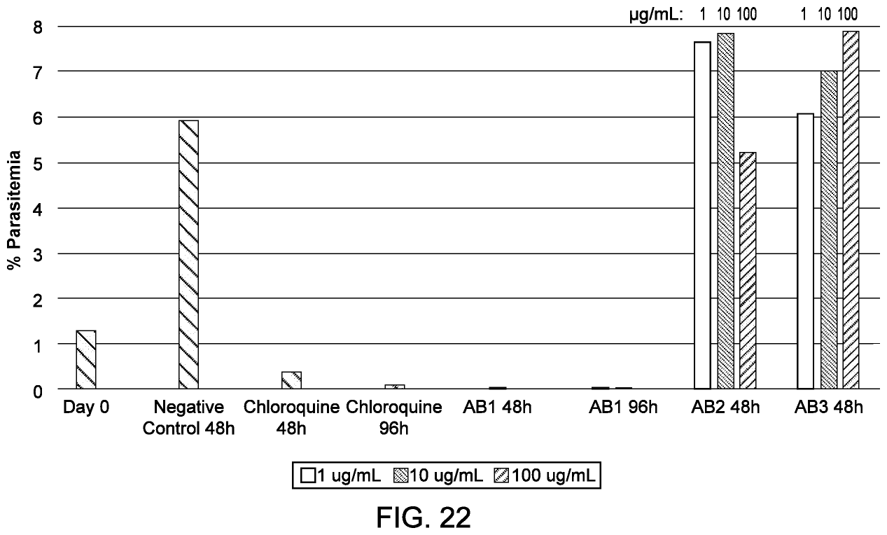

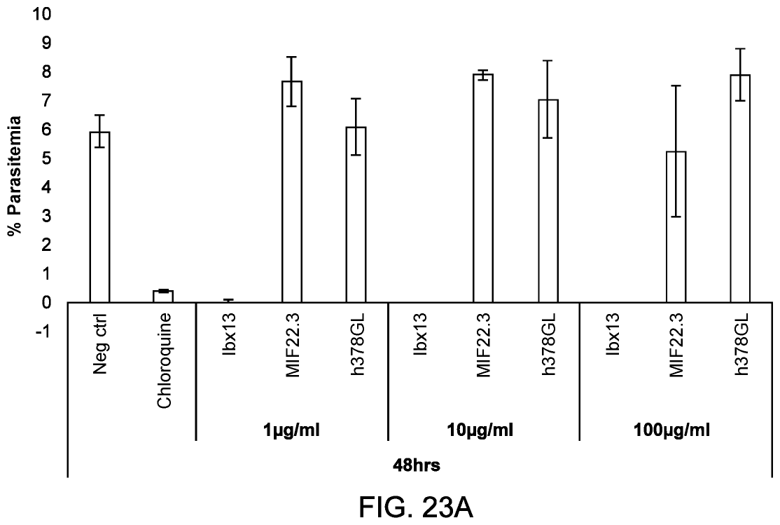

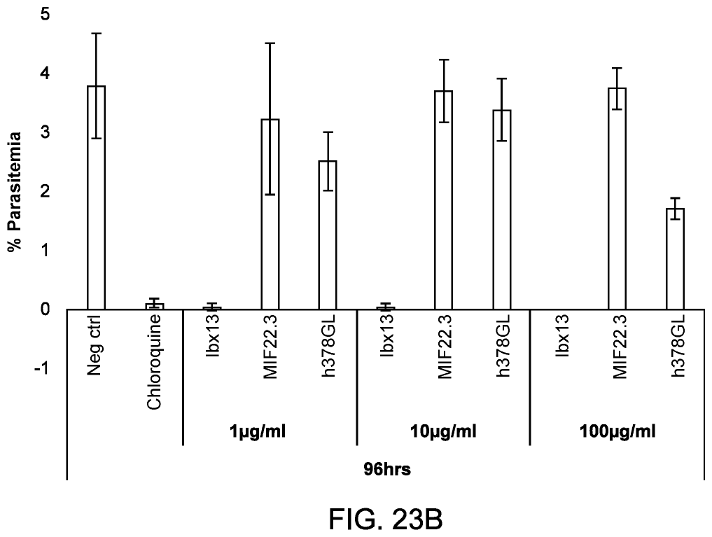

The results are shown in FIGS. 22 and 23 and Table 2.

In the positive control treatment, parasites grew normally after 48 hours, increasing from 1.3% to 5.9%. In the negative control, treatment with chloroquine killed about 93% of the parasites at 48 hours, and approximately 98% at 96 hours. While treatment with Ibx-13 resulted in ˜100% parasite inhibition, even at the lowest concentration tested, treatment with MIF-2-5 or VhH-Fc had no measurable effect. By 96 hours, parasites in the control, MIF-2-5, and VhH-Fc groups were dying due to very high parasitemia because the medium was not changed daily.

TABLE 2
Average parasitemia

| | Average |
|---|---|
| Test reagent | parasitemia |
| Day 0 | 1.296296296 |

| | 48 Hours | | 96 Hours | |
|---|---|---|---|---|
| | Average | | Average | |
| Test reagent | parasitemia | 1-3 St. Dev. | parasitemia | 1-3 St. Dev. |
| Negative | 5.918153926 | 0.566668 | 3.77439075 | 0.893404 |
| control | | | | |
| Chloroquine | 0.38513122 | 0.047377 | 0.096743308 | 0.078889 |
| Ibx-13 | 0.031836995 | 0.055143 | 0.032552083 | 0.056382 |
| (1 μg/mL) | | | | |
| Ibx-13 | 0 | 0 | 0.033134526 | 0.057391 |
| (10 μg/mL) | | | | |
| Ibx-13 | 0 | 0 | 0 | 0 |
| (100 μg/mL) | | | | |
| MIF-2-5 | 7.652795683 | 0.855565 | 3.222523593 | 1.286074 |
| (1 μg/mL) | | | | |
| MIF-2-5 | 7.855449078 | 0.172301 | 3.701698497 | 0.54011 |
| (10 μg/mL) | | | | |
| MIF-2-5 | 5.231480046 | 2.277871 | 3.740970126 | 0.35311 |
| (100 μg/mL) | | | | |
| VhH-Fc | 6.070595717 | 0.980876 | 2.508692048 | 0.496693 |
| (1 μg/mL) | | | | |
| VhH-Fc | 7.028640946 | 1.334009 | 3.376404601 | 0.529822 |
| (10 μg/mL) | | | | |
| VhH-Fc | 7.879652968 | 0.889477 | 1.702611895 | 0.17876 |
| (100 μg/mL) | | | | |

### Example 15—Antibody Drug Conjugate Assay on Malignant Melanoma Cells

2,500 A375 (malignant melanoma) cells were plated per well in a 96-well plate in complete growth medium (DMEM, 1000 FBS) and were allowed to adhere overnight. The next day, the old medium was replenished with 50 μl of fresh complete media and 25 μl of the Ibx-11, Ibx-13, or control antibodies (diluted in complete media). The antibodies were added in amounts ranging from 30 nM to 1.1 nM in 3-fold decreasing concentrations while the secondary ADC was kept constant at 30 nM. These mixtures were allowed to incubate for 5 minutes. 25 μl of the anti-Fc (human) secondary antibody drug conjugate reagent (diluted in complete media) was added to the wells and incubated for 72 hours at 37° C., in a 5.00% C02 incubator. After 72 hours, the viability was assessed using Viability Kit® according to the manufacturers protocol.

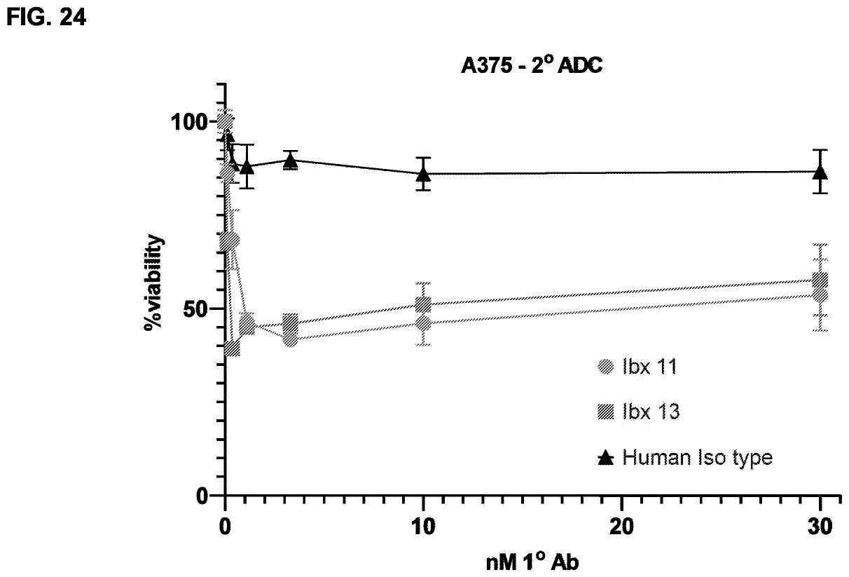

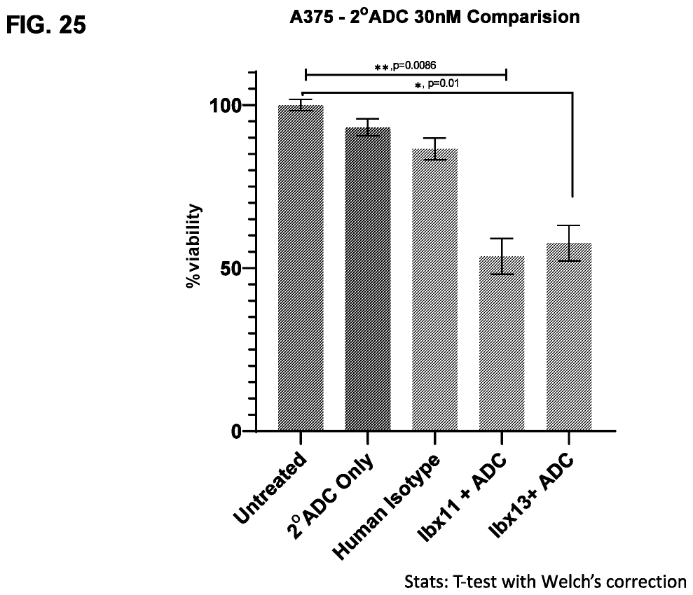

A375 cells show sensitivity to increasing doses up to 3.3 nM of the Primary antibodies Ibx-11 and Ibx-13. See FIGS. 24-25. The maximum effect observed was ˜55% loss of viability at 3.3 nM, with viability then tapering off at higher antibody concentrations. The secondary ADC itself at 30 nM may have a small effect compared to the untreated cells. Some effect of human isotype controls was also observed.

### Example 16—Antibody Drug Conjugate Assay on Prostate Cancer Cells

2,500 PC-3 (prostate cancer) cells were plated per well in a 96-well plate in complete growth medium (F-12K, 10% FBS) and were allowed to adhere overnight. The next day, the old medium was replenished with 50 μl of fresh complete media and 25 μl of the Ibx-11, Ibx-13, or control antibodies (diluted in complete media). The antibodies were added in amounts ranging from 30 nM to 1.1 nM in 3-fold decreasing concentrations while the secondary ADC was kept constant at 30 nM. These mixtures were allowed to incubate for 5 minutes. 25 μl of the anti-Fc (human) secondary antibody drug conjugate reagent (diluted in complete media) was added to the wells and incubated for 72 hours at 37° C., in a 5.0% CO2 incubator. After 72 hours, the viability was assessed using Viability Kit® according to the manufacturers protocol.

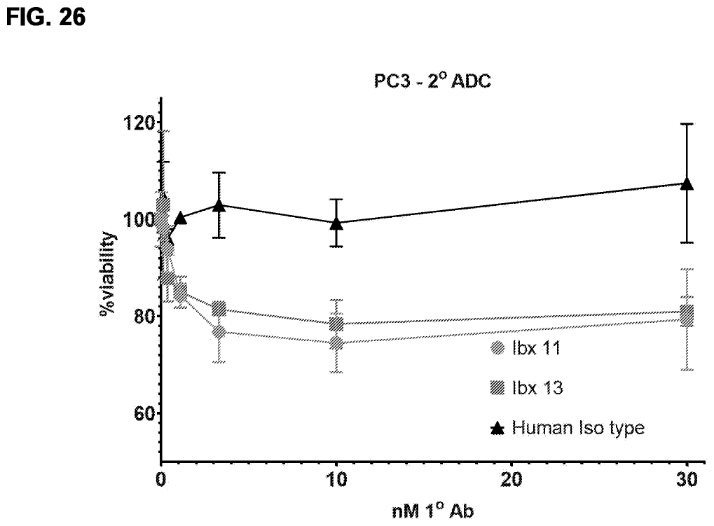

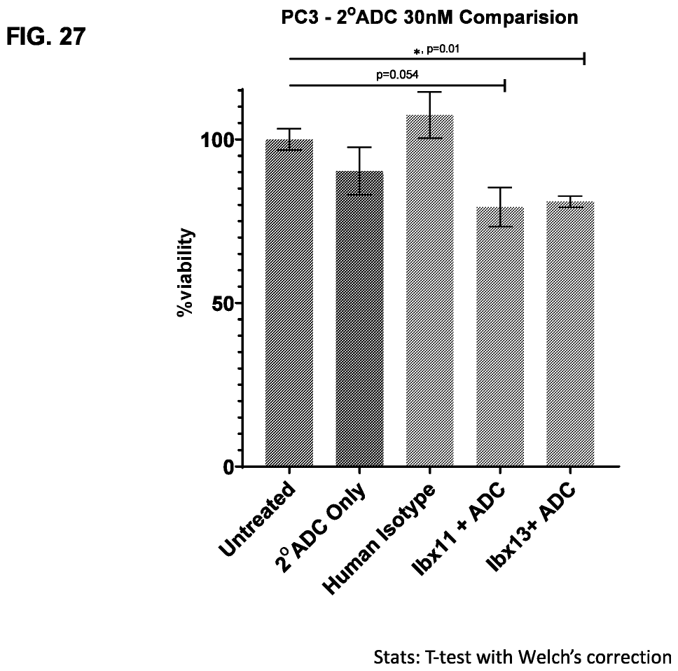

The PC3 cells may be less susceptible to the ADC assay than the A375 (malignant melanoma) cells taught in Example 15. See FIGS. 26 and 27. The maximum effect observed here was about a 25% loss of viability. This difference in viability may be due to the amount of surface CD147 present on the cells and the corresponding differences in the rate of endocytosis and the susceptibility of the cancer to the toxic payload used in the assay.

### REFERENCES

Watanabe et al. (2010). CD147/EMMPRIN Acts as a Functional Entry Receptor for Measles Virus on Epithelial Cells. Journal of Virology April 2010, 84 (9) 4183-4193.
Chenglong et al. (2020) Cyclophilin A and CD147: novel therapeutic targets for the treatment of COVID-19. Medicine in Drug Discovery, Volume 7:100056

1. An anti-CD147 antibody comprising a binding domain that comprises at least one CDR comprising at least 70% identity to an amino acid sequence selected from SEQ ID NOs: 16-39.

2. The anti-CD147 antibody of claim 1, wherein the anti-CD147 antibody comprises two CDRs, wherein each of the two CDRs independently comprise at least 70% identity to an amino acid sequence selected from SEQ ID NOs: 16-39.

3. The anti-CD147 antibody of claim 1, wherein the anti-CD147 antibody comprises three CDRs, wherein each of the three CDRs independently comprise at least 70% identity to an amino acid sequence selected from SEQ ID NOs: 16-39.

4. (canceled)

5. The anti-CD147 antibody of claim 1, wherein the anti-CD147 antibody comprises an amino acid sequence selected from SEQ ID NOs: 4-15.

6. The anti-CD147 antibody of claim 1, wherein the antibody is a full-length antibody, a monospecific antibody, a bispecific antibody, a trispecific antibody, an antigen-binding region, heavy chain, light chain, VhH, Vh, a CDR, a variable domain, scFv, Fc, Fv, Fab, F(ab)2, IgG, reduced IgG (rIgG), monospecific Fab2, bispecific Fab2, trispecific Fab3, diabody, bispecific diabody, trispecific triabody, minibody, nanobody, IgNAR, V-NAR, HcIgG, or a combination thereof.

7. The anti-CD147 antibody of claim 6, wherein the antibody is the VhH.

8. The anti-CD147 antibody of claim 1, wherein the antibody is a chimeric antigen receptor (CAR).

9. (canceled)

10. (canceled)

11. The anti-CD147 antibody of claim 1, wherein the anti-CD147 antibody binds to a CD147 or fragment thereof expressed on the surface of a cell.

12. (canceled)

13. The anti-CD147 antibody of claim 1, wherein the anti-CD147 antibody reduces or eliminates interaction between a virus and CD147.

14.-17. (canceled)

18. The anti-CD147 antibody of claim 13, wherein the virus is selected from the group consisting of: measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis.

19.-23. (canceled)

24. The anti-CD147 antibody of claim 22, wherein the cancer is selected from the group consisting of: breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

25.-28. (canceled)

29. The anti-CD147 antibody of claim 1, wherein the anti-CD147 antibody is humanized.

30.-40. (canceled)

41. An anti-CD147 antibody that comprises a CDR1, a CDR2, and a CDR3 region, wherein the CDR1 region is an amino acid sequence selected from the group consisting of: SEQ ID NO: 16-SEQ ID NO: 23, wherein the CDR2 region is an amino acid sequence selected from the group consisting of: SEQ ID NO: 24-SEQ ID NO: 31, wherein the CDR3 region is an amino acid sequence selected from the group consisting of: SEQ ID NO: 32-SEQ ID NO: 39, and wherein the anti-CD147 antibody comprises from 0 to 5 amino acid modifications in at least one of the CDR1, CDR, or CDR3 regions.

42.-45. (canceled)

46. A method of treatment, comprising administering an effective amount of a pharmaceutical composition comprising an anti-CD147 antibody, wherein the anti-CD147 antibody comprises a CDR1, a CDR2, and a CDR3 region, wherein the CDR1 region is an amino acid sequence selected from the group consisting of: SEQ ID NO: 16-SEQ ID NO: 23, wherein the CDR2 region is an amino acid sequence selected from the group consisting of: SEQ ID NO: 24-SEQ ID NO: 31, wherein the CDR3 region is an amino acid sequence selected from the group consisting of: SEQ ID NO: 32-SEQ ID NO: 39, and wherein the anti-CD147 antibody comprises from 0 to 5 amino acid modifications in at least one of the CDR1, CDR, or CDR3 regions.

47. The method of treatment of claim 46, wherein the administering is effective in reducing or eliminating an inflammatory or autoimmune disease.

48. The method of treatment of claim 47, wherein the inflammatory or autoimmune disease is selected from the group consisting of: rheumatoid arthritis, systemic lupus erythematosus (SLE), celiac disease, inflammatory bowel disease, Hashimoto's disease, Addison's disease, Grave's disease, type I diabetes, autoimmune thrombocytopenic purpura (ATP), idiopathic pulmonary fibrosis, idiopathic thrombocytopenia purpura (ITP), Crohn's disease, multiple sclerosis, and myasthenia gravis.

49. The method of treatment of claim 46, wherein the administering is effective in reducing or eliminating viral invasion of a cell by a virus.

50. The method of treatment of claim 49, wherein the virus is selected from the group consisting of: measles, coronavirus, SARS, MERS, infectious hematopoietic necrosis virus (IHNV), parvovirus, Herpes Simplex Virus, Hepatitis A virus, Hepatitis B virus, Hepatitis C virus, mumps virus, rubella virus, HIV, Influenza virus, Rhinovirus, Rotavirus A, Rotavirus B, Rotavirus C, Respiratory Syncytial Virus (RSV), Varicella zoster, Poliovirus, immunodeficiency virus (e.g. HIV), enveloped virus, RNA virus, and hepatitis.

51.-52. (canceled)

53. The method of treatment of claim 46, wherein the administering is effective in reducing or eliminating metastasis of a cancer.

54. The method of treatment of claim 53, wherein the cancer is selected from the group consisting of breast cancer, lung cancer, prostate cancer, ovarian cancer, brain cancer, liver cancer, cervical cancer, colon cancer, renal cancer, skin cancer, head & neck cancer, bone cancer, esophageal cancer, bladder cancer, uterine cancer, lymphatic cancer, stomach cancer, pancreatic cancer, testicular cancer, leukemia, acute lymphocytic leukemia (ALL), acute myelogenous leukemia (AML), chronic lymphocytic leukemia (CLL), chronic myelogenous leukemia (CML), and mantle cell lymphoma (MCL).

55. The method of treatment of claim 46, wherein the administering is effective in reducing or eliminating invasion of Plasmodium into a cell.

56.-62. (canceled)


SEQUENCE LISTING (42 sequences)

SEQ ID NO: 1 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 2 - protein, 389 aa, Mus musculus
MAAALLLALAFTLLSGQGACAAAGFLKAPLSQERWAGGSVVLHCEAVGSPIPEIQWWFEGNAPNDSCSQLWDGARLDRVHIHAAYRQHAASSLSVDGLTAEDTGTYECRASSDPDRNHLTRPPRVKWVRAQASVVVLEPGTIQTSVQEVNSKTQLTCSLNSSGVDIVGHRWMRGGKVLQEDTLPDLHTKYIVDADDRSGEYSCIFLPEPVGRSEINVEGPPRIKVGKKSEHSSEGELAKLVCKSDASYPPITDWFWFKTSDTGEEEAITNSTEANGKYVVVSTPEKSQLTISNLDVNVDPGTYVCNATNAQGTTRETISLRVRSRMAALWPFLGIVAEVLVLVTIIFIYEKRRKPDQTLDEDDPGAAPLKGSGTHMNDKDKNVRQRNAT

SEQ ID NO: 3 - protein, 388 aa, Rattus norvegicus
MAAALLLALAFTFLSGQGACAAAGFLKAPMSQEQWAGGSVVLHCEAVGSPMPEIQWWFEGNEPNDSCSQLWDGARLDRVHIHATYRQHAASTLSVDGLAAEDTGTYECRASSDPDRNHLTRPPRVKWVRAQASVVVLEPGTIVTSVQEVDSKTQLTCFLNSSGIDIVGHRWMRGGKVLQEDTLPDLQMKYTVDADDRSGEYSCIFLPEPVGRGNINVEGPPRIKVGKKSEHASEGEFVKLICKSEASHPPVDEWVWFKTSDTGDQTISNGTEANSKYVIISTPELSELIISDLDMNVDPGTYVCNATNSQGSARETISLRVRSRLAALWPFLGIVAEVLVLVTIIFIYEKRRKPDQTLDEDDPGAAPLKGSGSHLNDKDKNVRQRNAT

SEQ ID NO: 4 - protein, 364 aa, synthetic construct
  REGION 1..364: note = VHH Antibody-Fragment Crystallizable (Fc) Fusion Construct Ibx-11-Fc
QVRLEESGGGLVQAGGSLRLSCAASGLAFSIYGMGWFRQAPGKEREFVSVVDNTGSTTLYADSVKGRFTISRDNAKNTVYLQMSSLEPEDTAVYYCGAGPKNYLGSHEYAYWGQGTQVTVSSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 369 aa, synthetic construct
  REGION 1..369: note = VHH Antibody-Fragment Crystallizable (Fc) Fusion Construct Ibx-12-Fc
QVRLVESGGGLVQAGDSLRLSCQASGGTFTTYAMGWFRQAPGQEREFVSATTWSTGTSYYAYAADSVKGRFTTSRDNDNTVTLTMKRLKPDDTAVYYCSADPDGSDWAHSARQLRYWGQGTQVTVSSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 367 aa, synthetic construct
  REGION 1..367: note = VHH Antibody-Fragment Crystallizable (Fc) Fusion Construct Ibx-13-Fc
QVQLVESGGGLVQPGGSLRLSCTASEDTFSTYAIGWFRQAPGQGREFVAGITANGIATRYNDSLKDRFTISRDNAKNTVYLQMNSLQPEDTAVYSCGADTRRFSLPSTVHEIDYWGQGTQVTVSSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 362 aa, synthetic construct
  REGION 1..362: note = VHH Antibody-Fragment Crystallizable (Fc) Fusion Construct Ibx-14-Fc
QVQLVESGGGLVQAGDSLTLSCAASGRSLSSNAMGWFRQAPGKEREFVSGTTWHAGTQYYAYAADSVKGRFTTSRDNANTVSLQMKRLKPEDTAVYYCAAAVSGPGYDSWGQGTQVTVSSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 271 aa, synthetic construct
  REGION 1..271: note = VHH Antibody-Fragment Crystallizable (Fc) Fusion Construct Ibx-74 (dimer of IBX-11)
QVRLEESGGGLVQAGGSLRLSCAASGLAFSIYGMGWFRQAPGKEREFVSVVDNTGSTTLYADSVKGRFTISRDNAKNTVYLQMSSLEPEDTAVYYCGAGPKNYLGSHEYAYWGQGTQVTVSSEPKIPQPQPKPQPQPQPQPQPKPQPEPQVRLEESGGGLVQAGGSLRLSCAASGLAFSIYGMGWFRQAPGKEREFVSVVDNTGSTTLYADSVKGRFTISRDNAKNTVYLQMSSLEPEDTAVYYCGAGPKNYLGSHEYAYWGQGTQVTVSS

SEQ ID NO: 9 - protein, 281 aa, synthetic construct
  REGION 1..281: note = VHH Antibody-Fragment Crystallizable (Fc) Fusion Construct Ibx-75 (dimer of IBX-12)
QVRLVESGGGLVQAGDSLRLSCQASGGTFTTYAMGWFRQAPGQEREFVSATTWSTGTSYYAYAADSVKGRFTTSRDNDNTVTLTMKRLKPDDTAVYYCSADPDGSDWAHSARQLRYWGQGTQVTVSSEPKIPQPQPKPQPQPQPQPQPKPQPEPQVRLVESGGGLVQAGDSLRLSCQASGGTFTTYAMGWFRQAPGQEREFVSATTWSTGTSYYAYAADSVKGRFTTSRDNDNTVTLTMKRLKPDDTAVYYCSADPDGSDWAHSARQLRYWGQGTQVTVSS

SEQ ID NO: 10 - protein, 277 aa, synthetic construct
  REGION 1..277: note = VHH Antibody-Fragment Crystallizable (Fc) Fusion Construct Ibx-76 (dimer of IBX-13)
QVQLVESGGGLVQPGGSLRLSCTASEDTFSTYAIGWFRQAPGQGREFVAGITANGIATRYNDSLKDRFTISRDNAKNTVYLQMNSLQPEDTAVYSCGADTRRFSLPSTVHEIDYWGQGTQVTVSSEPKIPQPQPKPQPQPQPQPQPKPQPEPQVQLVESGGGLVQPGGSLRLSCTASEDTFSTYAIGWFRQAPGQGREFVAGITANGIATRYNDSLKDRFTISRDNAKNTVYLQMNSLQPEDTAVYSCGADTRRFSLPSTVHEIDYWGQGTQVTVSS

SEQ ID NO: 11 - protein, 267 aa, synthetic construct
  REGION 1..267: note = VHH Antibody-Fragment Crystallizable (Fc) Fusion Construct Ibx-77 (dimer of IBX-14)
QVQLVESGGGLVQAGDSLTLSCAASGRSLSSNAMGWFRQAPGKEREFVSGTTWHAGTQYYAYAADSVKGRFTTSRDNANTVSLQMKRLKPEDTAVYYCAAAVSGPGYDSWGQGTQVTVSSEPKIPQPQPKPQPQPQPQPQPKPQPEPQVQLVESGGGLVQAGDSLTLSCAASGRSLSSNAMGWFRQAPGKEREFVSGTTWHAGTQYYAYAADSVKGRFTTSRDNANTVSLQMKRLKPEDTAVYYCAAAVSGPGYDSWGQGTQVTVSS

SEQ ID NO: 12 - protein, 122 aa, synthetic construct
  REGION 1..122: note = Antibody Construct Ibx-11
QVRLEESGGGLVQAGGSLRLSCAASGLAFSIYGMGWFRQAPGKEREFVSVVDNTGSTTLYADSVKGRFTISRDNAKNTVYLQMSSLEPEDTAVYYCGAGPKNYLGSHEYAYWGQGTQVTVSS

SEQ ID NO: 13 - protein, 127 aa, synthetic construct
  REGION 1..127: note = Antibody Construct Ibx-12
QVRLVESGGGLVQAGDSLRLSCQASGGTFTTYAMGWFRQAPGQEREFVSATTWSTGTSYYAYAADSVKGRFTTSRDNDNTVTLTMKRLKPDDTAVYYCSADPDGSDWAHSARQLRYWGQGTQVTVSS

SEQ ID NO: 14 - protein, 125 aa, synthetic construct
  REGION 1..125: note = Antibody Construct Ibx-13
QVQLVESGGGLVQPGGSLRLSCTASEDTFSTYAIGWFRQAPGQGREFVAGITANGIATRYNDSLKDRFTISRDNAKNTVYLQMNSLQPEDTAVYSCGADTRRFSLPSTVHEIDYWGQGTQVTVSS

SEQ ID NO: 15 - protein, 120 aa, synthetic construct
  REGION 1..120: note = Antibody Construct Ibx-14
QVQLVESGGGLVQAGDSLTLSCAASGRSLSSNAMGWFRQAPGKEREFVSGTTWHAGTQYYAYAADSVKGRFTTSRDNANTVSLQMKRLKPEDTAVYYCAAAVSGPGYDSWGQGTQVTVSS

SEQ ID NO: 16 - protein, 10 aa, synthetic construct
  REGION 1..10: note = Ibx-11 CDR1
GLAFSIYGMG

SEQ ID NO: 17 - protein, 10 aa, synthetic construct
  REGION 1..10: note = Ibx-12 CDR1
GGTFTTYAMG

SEQ ID NO: 18 - protein, 10 aa, synthetic construct
  REGION 1..10: note = Ibx-13 CDR1
EDTFSTYAIG

SEQ ID NO: 19 - protein, 10 aa, synthetic construct
  REGION 1..10: note = Ibx-14 CDR1
GRSLSSNAMG

SEQ ID NO: 20 - protein, 14 aa, synthetic construct
  REGION 1..14: note = Ibx-11 (flanking framework) CDR1
ASGLAFSIYGMGWF

SEQ ID NO: 21 - protein, 14 aa, synthetic construct
  REGION 1..14: note = Ibx-12 (flanking framework) CDR1
ASGGTFTTYAMGWF

SEQ ID NO: 22 - protein, 14 aa, synthetic construct
  REGION 1..14: note = Ibx-13 (flanking framework) CDR1
ASEDTFSTYAIGWF

SEQ ID NO: 23 - protein, 14 aa, synthetic construct
  REGION 1..14: note = Ibx-14 (flanking framework) CDR1
ASGRSLSSNAMGWF

SEQ ID NO: 24 - protein, 17 aa, synthetic construct
  REGION 1..17: note = Ibx-11 CDR2
VVDNTGSTTLYADSVKG

SEQ ID NO: 25 - protein, 20 aa, synthetic construct
  REGION 1..20: note = Ibx-12 CDR2
ATTWSTGTSYYAYAADSVKG

SEQ ID NO: 26 - protein, 17 aa, synthetic construct
  REGION 1..17: note = Ibx-13 CDR2
GITANGIATRYNDSLKD

SEQ ID NO: 27 - protein, 20 aa, synthetic construct
  REGION 1..20: note = Ibx-14 CDR2
GTTWHAGTQYYAYAADSVKG

SEQ ID NO: 28 - protein, 21 aa, synthetic construct
  REGION 1..21: note = Ibx-11 (flanking framework) CDR2
VSVVDNTGSTTLYADSVKGRF

SEQ ID NO: 29 - protein, 24 aa, synthetic construct
  REGION 1..24: note = Ibx-12 (flanking framework) CDR2
VSATTWSTGTSYYAYAADSVKGRF

SEQ ID NO: 30 - protein, 21 aa, synthetic construct
  REGION 1..21: note = Ibx-13 (flanking framework) CDR2
VAGITANGIATRYNDSLKDRF

SEQ ID NO: 31 - protein, 24 aa, synthetic construct
  REGION 1..24: note = Ibx-14 (flanking framework) CDR2
VSGTTWHAGTQYYAYAADSVKGRF

SEQ ID NO: 32 - protein, 15 aa, synthetic construct
  REGION 1..15: note = Ibx-11 CDR3
GAGPKNYLGSHEYAY

SEQ ID NO: 33 - protein, 18 aa, synthetic construct
  REGION 1..18: note = Ibx-12 CDR3
SADPDGSDWAHSARQLRY

SEQ ID NO: 34 - protein, 18 aa, synthetic construct
  REGION 1..18: note = Ibx-13 CDR3
GADTRRFSLPSTVHEIDY

SEQ ID NO: 35 - protein, 11 aa, synthetic construct
  REGION 1..11: note = Ibx-14 CDR3
AAAVSGPGYDS

SEQ ID NO: 36 - protein, 19 aa, synthetic construct
  REGION 1..19: note = Ibx-11 (flanking framework) CDR3
YCGAGPKNYLGSHEYAYWG

SEQ ID NO: 37 - protein, 22 aa, synthetic construct
  REGION 1..22: note = Ibx-12 (flanking framework) CDR3
YCSADPDGSDWAHSARQLRYWG

SEQ ID NO: 38 - protein, 22 aa, synthetic construct
  REGION 1..22: note = Ibx-13 (flanking framework) CDR3
SCGADTRRFSLPSTVHEIDYWG

SEQ ID NO: 39 - protein, 15 aa, synthetic construct
  REGION 1..15: note = Ibx-14 (flanking framework) CDR3
YCAAAVSGPGYDSWG

SEQ ID NO: 40 - protein, 15 aa, synthetic construct
  REGION 1..15: note = linker
GGGGSGGGGSGGGGS

SEQ ID NO: 41 - protein, 27 aa, synthetic construct
  REGION 1..27: note = linker
EPKIPQPQPKPQPQPQPQPQPKPQPEP

SEQ ID NO: 42 - protein, 8 aa, synthetic construct
  REGION 1..8: note = flag tag
DYKDDDDK